(19) United States
(12) Patent Application Publication
(10) Pub. No.: US 2018/0078656 A1
(43) Pub. Date: Mar. 22, 2018

(54) CRYPTOPHYCIN-BASED ANTIBODY-DRUG CONJUGATES WITH NOVEL SELF-IMMOLATIVE LINKERS

(71) Applicant: EXIRIS S.R.L., Roma (IT)

(72) Inventors: M. Christian STEINKUHLER, Roma (IT); M. Paola GALLINARI, Roma (IT); Bianca OSSWALD, Bielefeld (DE); Norbert SEWALD, Bielefeld (DE); Markus RITZEFELD, Bielefeld (DE); Marcel FRESE, Enger (DE); Eduard FIGUERAS, Bielefeld (DE); Lilla PETHÖ, Pécs (HU)

(21) Appl. No.: 15/558,533

(22) PCT Filed: Mar. 15, 2016
(86) PCT No.: PCT/EP2016/055599

§ 371 Date: Sep. 14, 2017

(30) Foreign Application Priority Data
Mar. 17, 2015 (EP) 15159327.4

(51) Int. Cl. A61K 47/68; C07K 16/32; C07K 16/30

(52) U.S. Cl. CPC A61K 47/6889; A61K 47/6811; A61K 47/6871; C07K 16/3046; A61K 47/6863; C07K 16/32; C07K 16/3015; A61K 47/6855

(57) ABSTRACT

The present invention relates to antibody- or peptide-drug conjugate compounds where one or more cryptophycin derivatives (macrocyclic depsipeptide) are covalently attached by a self-immolative linker which binds to one or more tumor-associated antigens or cell-surface receptors. The linker contains a cleavage site for proteases and a dipeptide unit able to form a diketopiperazine. These compounds may be useful in methods of diagnosis or treatment of cancer, and other diseases and disorders, such as immune or infective diseases.

### FIELD OF THE INVENTION

The present invention relates to the field of medicinal chemistry, in particular to compounds with anti-cancer activity and more specifically to antibodies conjugated with chemotherapeutic macrocyclic depsipeptide drugs or toxins. The invention also relates to the above compounds for use in in vitro, in situ, and in vivo diagnosis or treatment of mammalian cells or subjects affected by cancer, an autoimmune disease, or an infectious disease.

### BACKGROUND OF THE INVENTION

Antibody therapy has been established for the targeted treatment of patients with cancer, immunological and angiogenic disorders. For this purpose, therapeutic monoclonal antibodies (TMAs) have been developed (Firer M. A. J. Hematol. Oncol. 2012, 5, 70; Mullard, A. Nature Rev. Drug Discov. 2013, 12, 329). However, many TMAs that showed promise in preclinical studies when used on their own failed in the clinic because of insufficient cancer cell toxicity.

Monoclonal antibody therapy has been established for the targeted treatment of patients with cancer, immunological and angiogenic disorders. An example of successful antibody therapy is HERCEPTIN® (trastuzumab), a recombinant DNA-derived humanized monoclonal antibody that selectively binds with high affinity to the extracellular domain of the human epidermal growth factor receptor 2 protein, HER2 (ErbB2) (U.S. Pat. No. 5,821,337; U.S. Pat. No. 6,054,297; U.S. Pat. No. 6,407,213; U.S. Pat. No. 6,639,055; Coussens L, et al (1985) Science 230:1132-9; Slamon D J, et al (1989) Science 244:707-12). Although HERCEPTIN is a breakthrough in treating patients with ErbB2-overexpressing breast cancers that have received extensive prior anti-cancer therapy, the majority of the patients in this population fail to respond or respond only poorly to HERCEPTIN treatment.

To overcome such a problem, there has been an exponential growth in the field of antibody-drug conjugates (ADCs), where a monoclonal antibody (mAb, e.g. trastuzumab) is linked to a highly cytotoxic drug. ADCs gain more and more importance in the therapy of cancer. Three ADCs have been approved by the US FDA: Mylotarg® (gemtuzumab ozogamicin, Wyeth Pharmaceuticals), Adcetris® (brentuximab vedotin, Seattle Genetics), and Kadcyla® (ado-trastuzumab emtansin, Roche), while 35 ADCs are currently in clinical trials. Tumor selectivity is displayed by the mAb whereas the linker is responsible for the release of the drug, the stability and the overall solubility under physiological conditions.

Mylotarg® is composed of a hu CD33 antibody linked to calicheamicin and was approved in 2000 for the treatment of acute myeloid leukemia, but withdrawn in 2010. Adcetris® (brentuximab vedotin, Seattle Genetics, Formula 1, below) was approved in 2011 and is composed of monomethyl auristatin E (MMAE) connected to an antibody against CD30, which is expressed in classical Hodgkin lymphoma (HL) and systemic anaplastic large cell lymphoma (sALCL), Kadcyla® (ado-trastuzumab emtansin, Roche, Formula 2, below) was approved in 2013 for treatment of refractory human epidermal growth factor receptor 2 (HER2) positive metastatic or locally advanced breast cancer and contains the monoclonal antibody trastuzumab (Herceptin) linked to the cytotoxic agent mertansine (DM1).

Ado-trastuzumab Emtansin (Kadcyla®) and Brentuximab Vedotin (Adcetris®) are the only two ADCs which are currently approved by the US Food and Drug Administration (FDA) and European Medicine Agency (EMA).

The extraordinary activity of cryptophycins has been acknowledged in some patents lately. Endocyte patented the cryptophycin folate conjugate (Formula ₃) equipped with a cleavable disulfide linker. Such folate conjugates target cancer cell lines overexpressing folate receptors (US2009/002993).

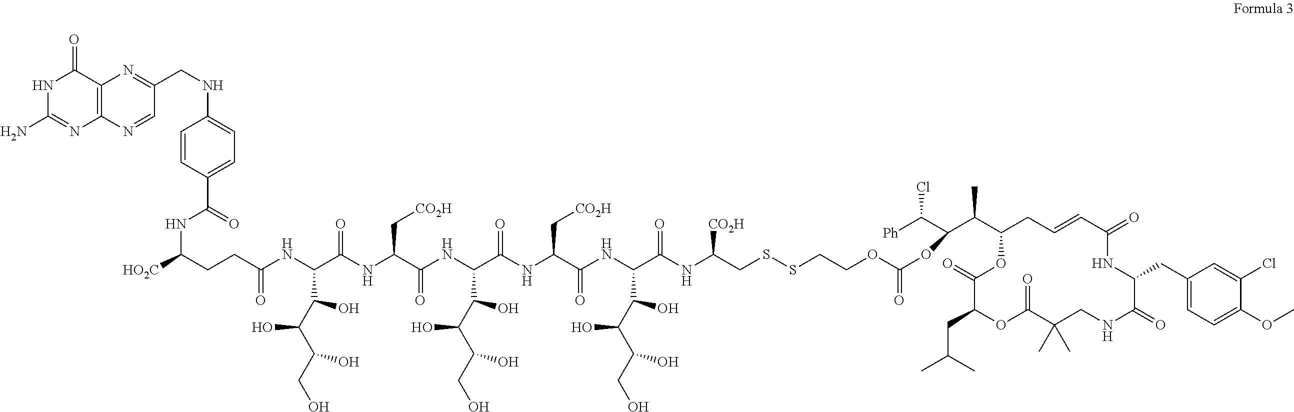 [SMILES: COc1ccc(C[C@H]2NC(=O)/C=C/C[C@@H]([C@H](C)[C@@H](OC(=O)OCCSSC[C@@H](NC(=O)[C@@H](NC(=O)[C@H](CC(=O)O)NC(=O)[C@@H](NC(=O)[C@H](CC(=O)O)NC(=O)[C@@H](NC(=O)CC[C@H](NC(=O)c3ccc(NCc4cnc5nc(N)[nH]c(=O)c5n4)cc3)C(=O)O)[C@@H](O)[C@H](O)[C@H](O)CO)[C@@H](O)[C@H](O)[C@H](O)CO)[C@@H](O)[C@H](O)[C@H](O)CO)C(=O)O)[C@@H](Cl)c3ccccc3)OC(=O)[C@H](CC(C)C)OC(=O)C(C)(C)CNC2=O)cc1Cl]

Sanofi-Aventis published two patents disclosing data on cryptophycin antibody-drug conjugates (ADCs) very recently (US2011/001052; FR2947269). Mainly cryptophycin derivatives with a modified aryl substituent were selected to obtain cryptophycins with suitable functionalities (Formula 4).

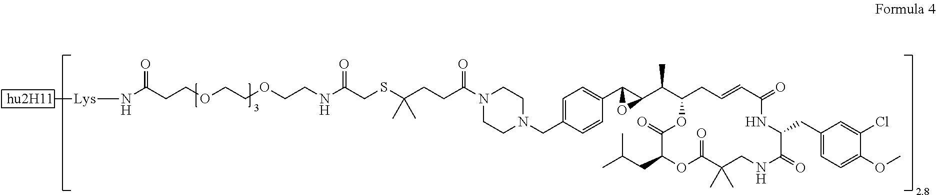

Different linker types, among them a cleavable disulfide-, a PEG-, a thioether- and a thiazole linker were used. In all cases, the linker was connected to hu2H11, a monoclonal antibody targeting the EphA2 receptor. The IC₅₀ values for two cryptophycin ADCs were also reported against the human breast cancer cell-line MDA-MB-231. The ADC shown in Formula 4 contains a sterically hindered thioether linker and shows an IC₅₀ value of 0.710 nM. Another ADC contains a sterically hindered disulfide linker (IC₅₀: 0.150 nM). The substituted cryptophycins were reported to loose cytotoxic activity on drug resistant cells.

In contrast, unconjugated Cryptophycins (Formula 5) are reported to have very high cytotoxicity even against multi-drug resistant (MDR) cancer cells. Cryptophycin-52 displays very high activity against multi-drug resistant (MDR) tumour cell lines. Many MDR tumour cells express P glycoprotein (P-gp), which acts as an efflux pump. As the rational design of P-gp resistant drugs is still difficult, this activity is another valuable feature of cryptophycins.

The first representative was isolated from cyanobacteria Nostoc sp. in 1990. Their bioactivity is based on their interaction with the protein tubulin. Cryptophycins were found to induce apoptosis due to inhibition of the microtubule dynamics. Consequently, cryptophycin analogues are considered as potential antitumor agents.

Cryptophycin-52 (LY355703) passed clinical phase I studies, but subsequent clinical phase II studies were discontinued because of lacking efficacy in vivo, high neurotoxicity, and dose-limiting toxicity at the chosen doses.

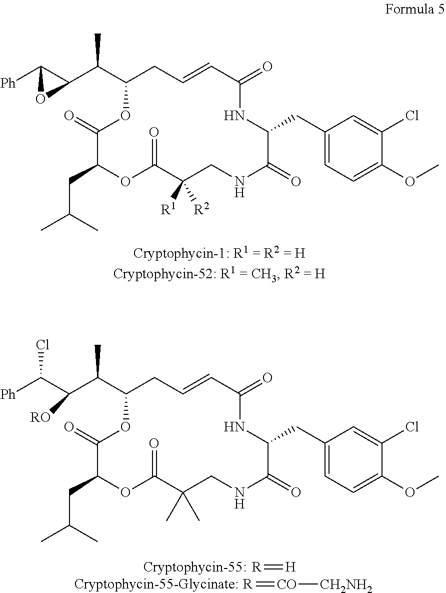

Only few ADCs have been currently approved for the therapy of cancer. A higher diversity of ADCs is necessary to cope with drug resistance.

Up to now no cryptophycin-based ADC has been approved and no cryptophycin-55-glycinate has been described for conjugation to an antibody

All described cryptophycin conjugates are only accessible by long synthetic routes (see for example W02011/001052). However, Sewald and co-workers described short and efficient synthetic routes (Nat. Prod. Rep. 2013, 30, 924-940).

There is still the strong need to provide drug conjugates, in particular antibody-drug conjugates (also commonly referred as ADCs) and peptide-drug conjugates (also commonly referred as PDCs) for safe use in therapy of diseases, in particular cancer, immunological diseases, infective diseases.

There is also still the strong need to provide drug conjugates, in particular antibody-drug conjugates (also commonly referred as ADCs) and peptide-drug conjugates (also commonly referred as PDCs) for use in diagnostics of diseases, in particular cancer, immunological diseases, infective diseases.

Another strong need is to provide drug conjugates, in particular antibody-drug conjugates (also commonly referred as ADCs) and peptide-drug conjugates (also commonly referred as PDCs) for use in therapy of cancer, immunological diseases, and infective diseases that develop multidrug resistance.

### DESCRIPTION OF THE INVENTION

It has now been found that conjugates where the chlorohydrin cryptophycin-55 is linked to a monoclonal antibody or a peptide across a self-immolative, peptide-derived linker are highly efficient against tumour cells.

The conjugates according to the present invention bind to one or more tumor-associated antigens or cell-surface receptors, therefore, they can be used in the diagnosis or treatment of those diseases which can be diagnosed or treated thanks to the binding to tumor-associated antigens or cell-surface receptors, proteins or antigens.

An object of the present invention is a conjugate of formula (I)

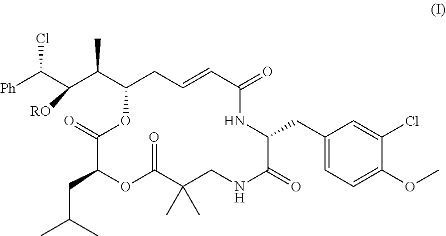

wherein R is —CO—CH₂—X-(A)ₙ-B

wherein X is selected from the group consisting of NR^{a}, wherein R^{a} is selected from a group consisting of H and C₁-C₁₀ alkyl, and O;

A is a self-immolative linker,

n is 0 or 1;

B is selected from the group consisting of a peptide, a polypeptide/protein, and an antibody

and the pharmaceutically acceptable salts thereof.

Another object of the present invention is a pharmaceutical composition comprising a conjugate as above described as active ingredient in admixture with at least one pharmaceutically acceptable vehicle and/or excipient.

Another object of the present invention is the conjugate as above described, for use as a medicament.

Another object of the present invention is the conjugate as above described, for use as a diagnostic.

These and other objects of the present invention will be now described in detail also by means of examples and Figures.

In the Figures:

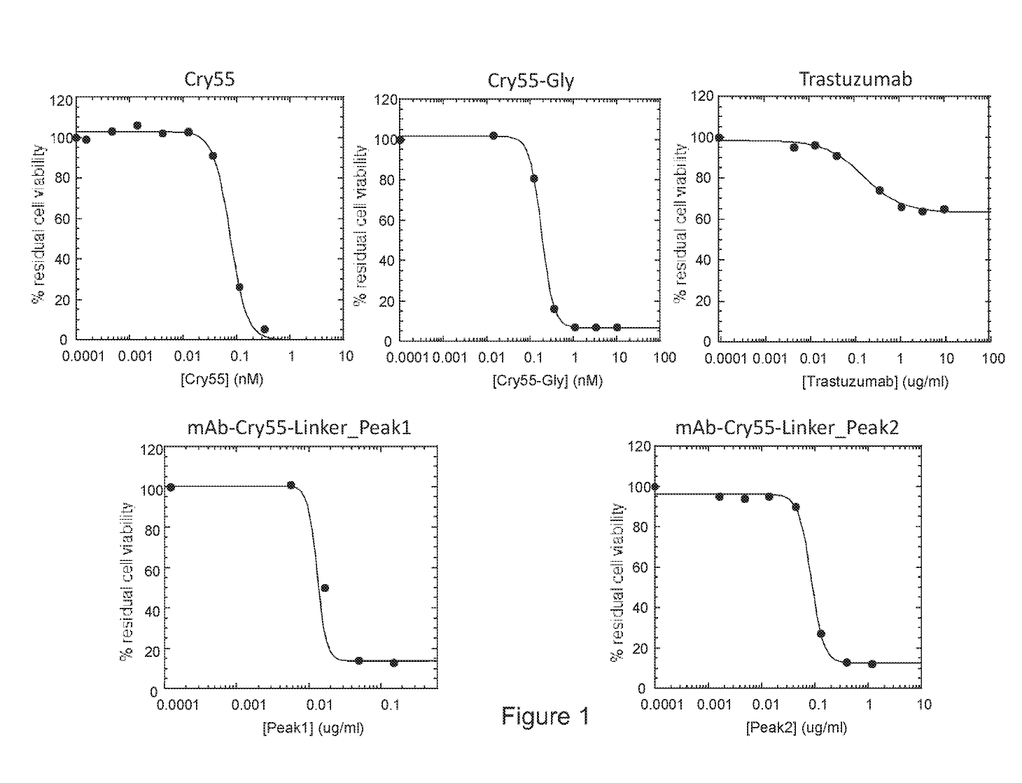

FIG. 1: shows cell viability assay after treatment of HER2-positive SK-BR3 cells with the compounds indicated in the panels.

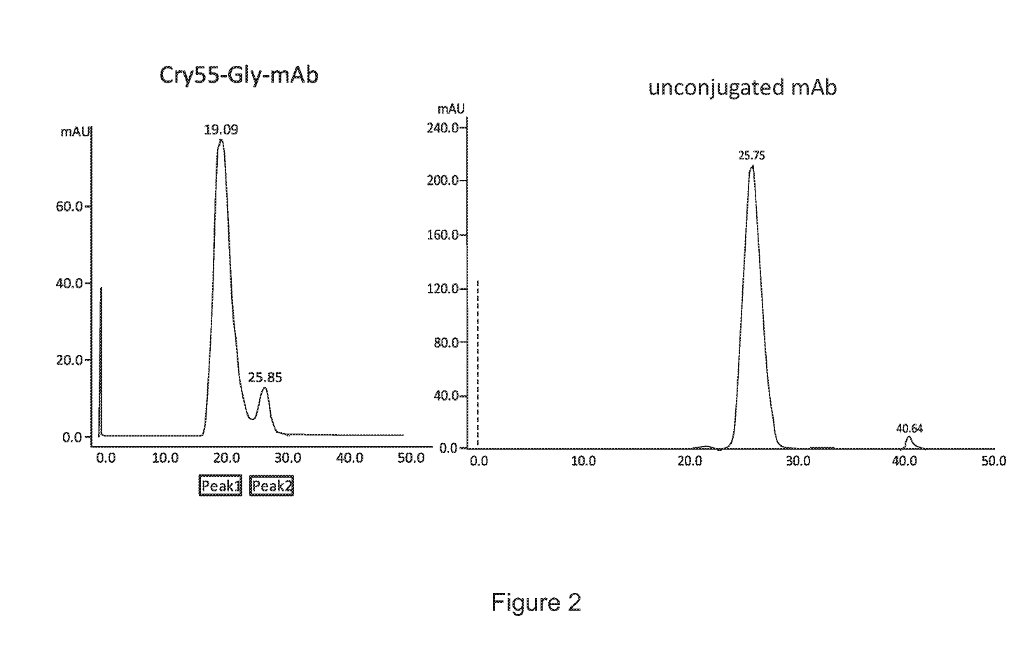

FIG. 2: shows purification of the drug linker-conjugated antibody wherein, peak 2 corresponds to the monomeric form and peak 1 corresponds to an oligomeric form of the cryptophycin-55-glycinate-conjugated antibody (FIG. 2, left panel), as assessed by comparison with suitable protein MW standards (not shown) and the purified unconjugated antibody (FIG. 2, right panel).

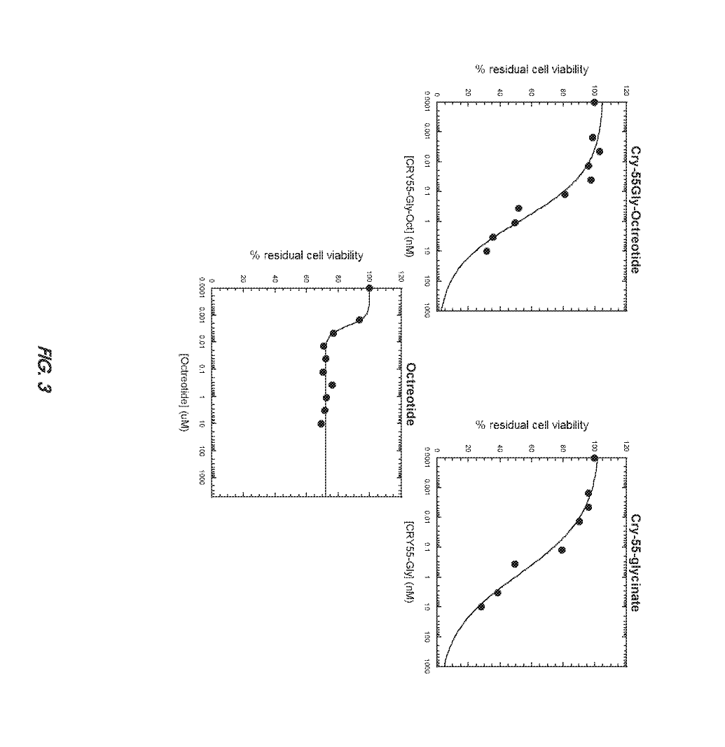

FIG. 3: shows residual cell viability plots obtained upon treatment of the AR42J cells with the conjugate of the present invention with octreotide.

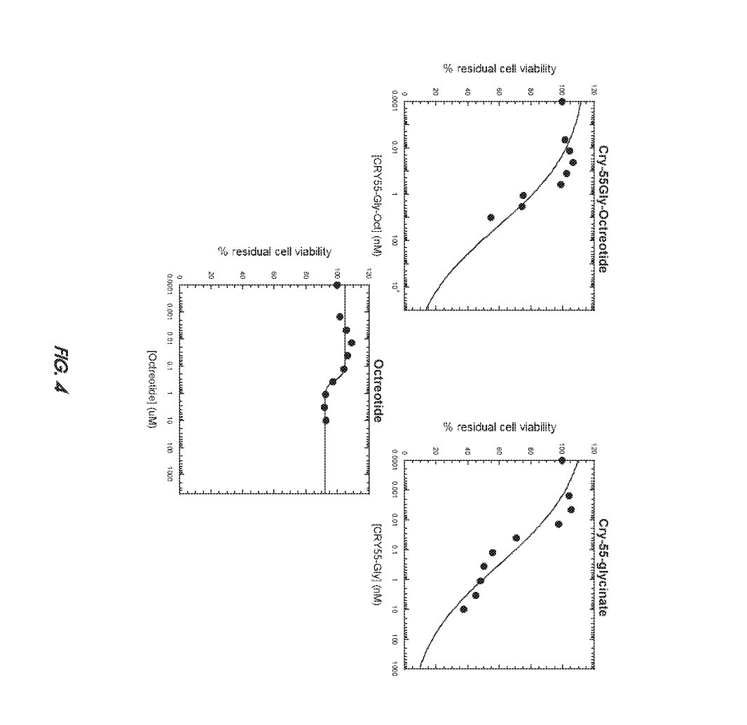

FIG. 4: shows residual cell viability plots obtained upon treatment of the MCF7 cells with the conjugate of the present invention with octreotide.

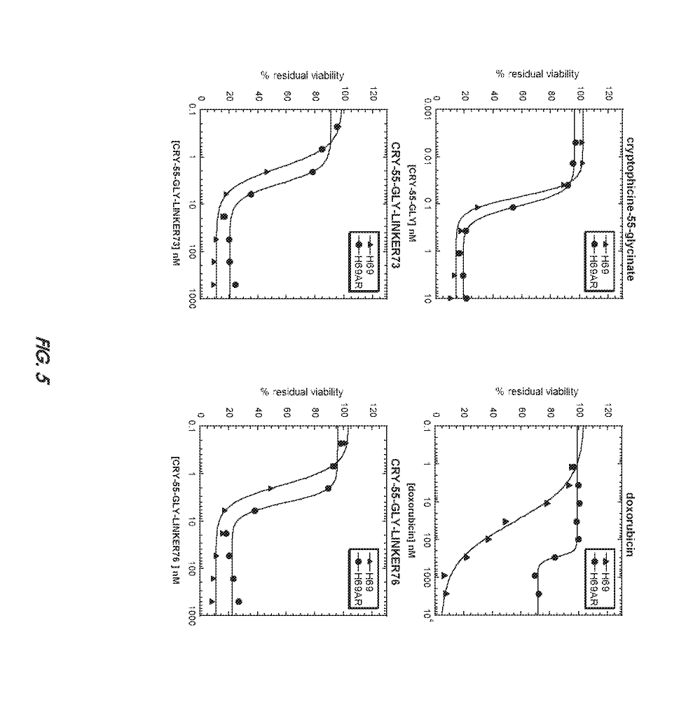

FIG. 5: shows cryptophycin or cryptophycin drug linkers cytotoxicity on wild type and drug resistant H69AR cell line. In the Figure, linker 73 is shown in Example 6, and linker 76 is shown in Example 5.

### DETAILED DESCRIPTION OF THE INVENTION

The conjugate according to the present invention can be a drug conjugate with an antibody, preferably a monoclonal antibody, or with a peptide, polypeptide or protein.

Antibodies, in particular monoclonal antibodies are well known in therapy, in particular for conjugation with a drug.

The conjugate according to the present invention can be a drug conjugate with an antibody, preferably a monoclonal antibody, in which case it is also briefly referred to as ADC, or with a peptide, polypeptide or protein, in which case is also briefly referred to as PDC.

Peptides are well known in therapy, in particular for conjugation with a drug. Peptides, polypeptides or proteins according to the present invention can comprise natural amino acids, either in L- or D- or both configuration, as well as artificial peptides.

In a preferred embodiment of the present invention, in the conjugate, the antibody is a monoclonal antibody, or a nanobody. According to a further embodiment of the present invention, the nanobody is selected from the group consisting of single-domain antibody and camelid antibody).

In a particularly preferred embodiment of the present invention, in the antibody drug conjugate (ADC) the monoclonal antibody is selected from the group consisting of trastuzumab, gemtuzumab, brentuximab, rituximab, cetuximab, panitumumab, ofatumumab, obinutuzumab, pertuzumab.

In a preferred embodiment of the present invention, the peptide B binds to somatostatin receptors. In a particularly preferred embodiment of the present invention, the peptide is selected from the group consisting of octreotide, pasireotide or lanreotide.

In an embodiment of the present invention, in the conjugate, the self-immolative linker A is a moiety of formula (II)

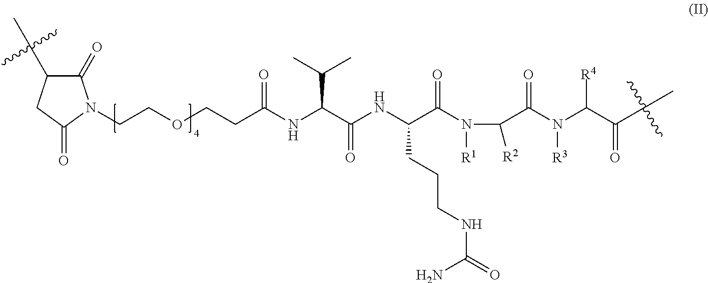

wherein R¹ is selected from the group consisting of H, (C₁-C₁₀) alkyl, R², optionally together with R¹, is the residue of an amino acid side chain, R³ is selected from the group consisting of H, (C₁-C₁₀) alkyl, R⁴, optionally together with R³, is the residue of an amino acid side chain.

In a preferred embodiment, in the conjugate, the self-immolative linker A is selected from the group consisting of

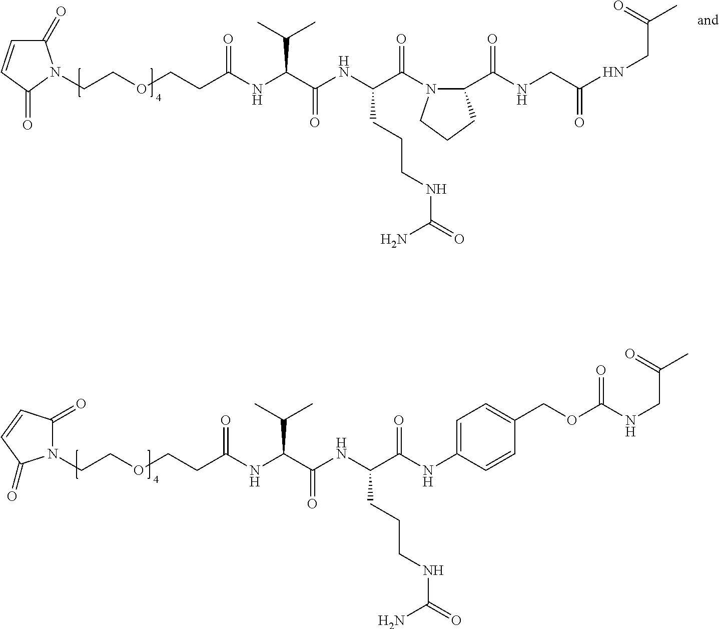

In another embodiment of the present invention, the conjugate has formula (III)

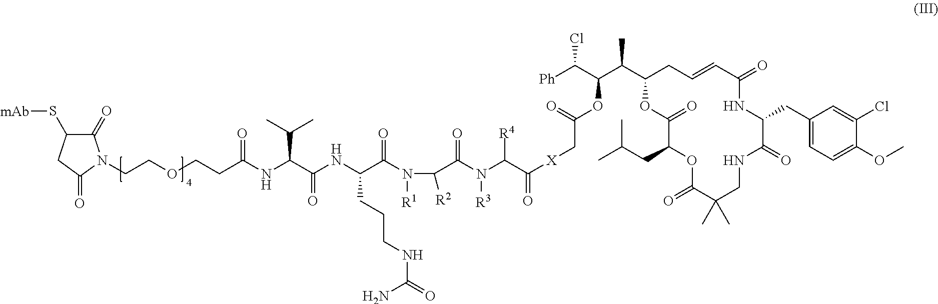

wherein R¹, R², R³ and R⁴ are as defined above, X is selected from the group consisting of NH, N—(C₁-C₁₀) alkyl, O; mAb represents a monoclonal antibody or a nanobody, or a peptide or a polypeptide.

In another embodiment of the present invention, the conjugate has formula (IV)

In another embodiment of the present invention, the conjugate has formula (V)

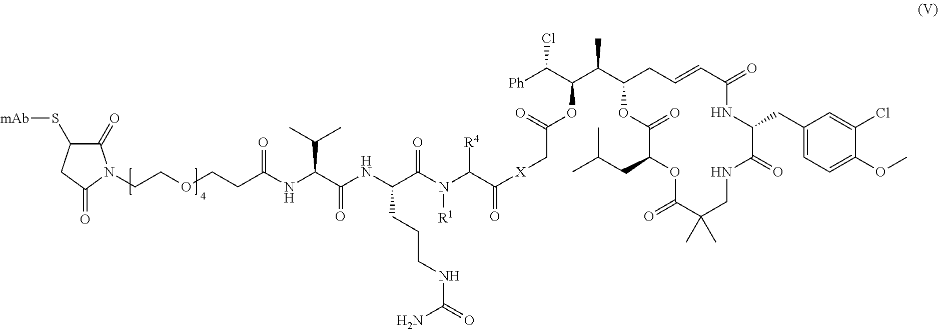

wherein R¹ is selected from the group consisting of H, (C₁-C₁₀) alkyl, R⁴, optionally together with R¹, is the residue of an amino acid side chain; X is selected from the group consisting of NH, N—(C₁-C₁₀) Alkyl-O; mAb represents a monoclonal antibody, a peptide or a polypeptide.

In another embodiment of the present invention, the conjugate has formula (VI)

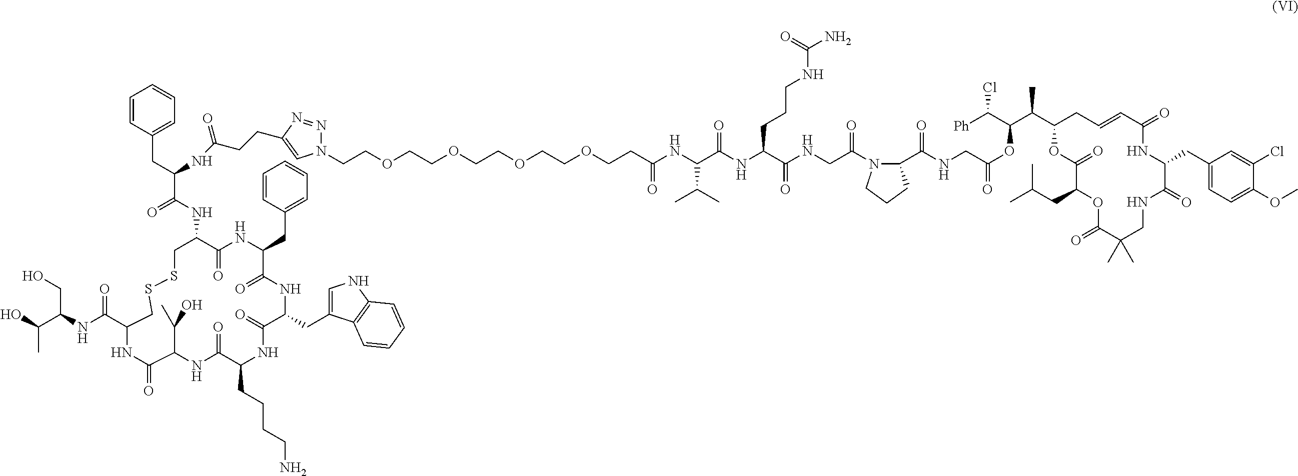

In an embodiment of the present invention, the conjugate is for use for the therapeutic treatment of a disease selected from the group consisting of cancer, autoimmune disease and infective disease.

In an embodiment of the present invention, the conjugate is for use for the diagnosis of a disease selected from the group consisting of cancer, autoimmune disease and infective disease. Other than in vivo, the conjugates according to the present invention can be used also for an in vitro or in situ diagnosis.

With the term C₁-C₁₀ alkyl, a linear or branched alkyl is intended. Examples of alkyl are methyl, ethyl, propyl, butyl, pentyl, hexyl, heptyl, octyl nonyl, decyl and all the isomers thereof.

According to the present invention, pharmaceutically acceptable salts are well-known in the art and do not need specific disclosure. The skilled person knows whether a salt is suitable to the purpose of the present invention and can resort to the broad reference of literature, manuals, handbooks, etc. for example Pharmaceutical Salts: Properties, Selection, and Use, P. Heinrich Stahl (Editor), Camille G. Wermuth (Editor), Wiley.

In a preferred aspect of the present invention, cryptophycin-55 glycinates or cryptophycin glycolates are used. They show cytotoxicity, which is not substantially lower than that of cryptophycin-55 (Table 1) and, additionally, have the benefit of a functional group which is suitable for conjugation. Via this function, cryptophycin-55-glycinate or cryptophycin-55-glycolate can be conjugated to a monoclonal antibody (for example, trastuzumab shows high selectivity towards the HER2-receptor which is overexpressed in 20-25% of all breast tumors) via a protease cleavable, self-immolative linker.

Self-immolative linkers are well-known in the design of drug-conjugates, in particular antibody-drug conjugates (ADC) and peptide-drug conjugates, see for example U.S. Pat. No. 6,214,345 and the related references; or Carl P. L. et al. (1982). The linker is composed of an attachment site (maleimide, active ester, etc.) which is reactive for functional groups present in proteins (thiols, amines, etc.), a hydrophilic spacer (e.g. ethylene glycol based), a protease cleavage site (e.g. Val-Cit), and a dipeptide (Scheme 2) or tripeptide unit (Scheme 1) able to release cryptophycin-55, cryptophycin-55 glycinate, cryptophycin-55 glycolate, or other peptidomimetic cryptophycin-55 derivatives. Cryptophycin-55 is assumed to be converted to cryptophycin-52.

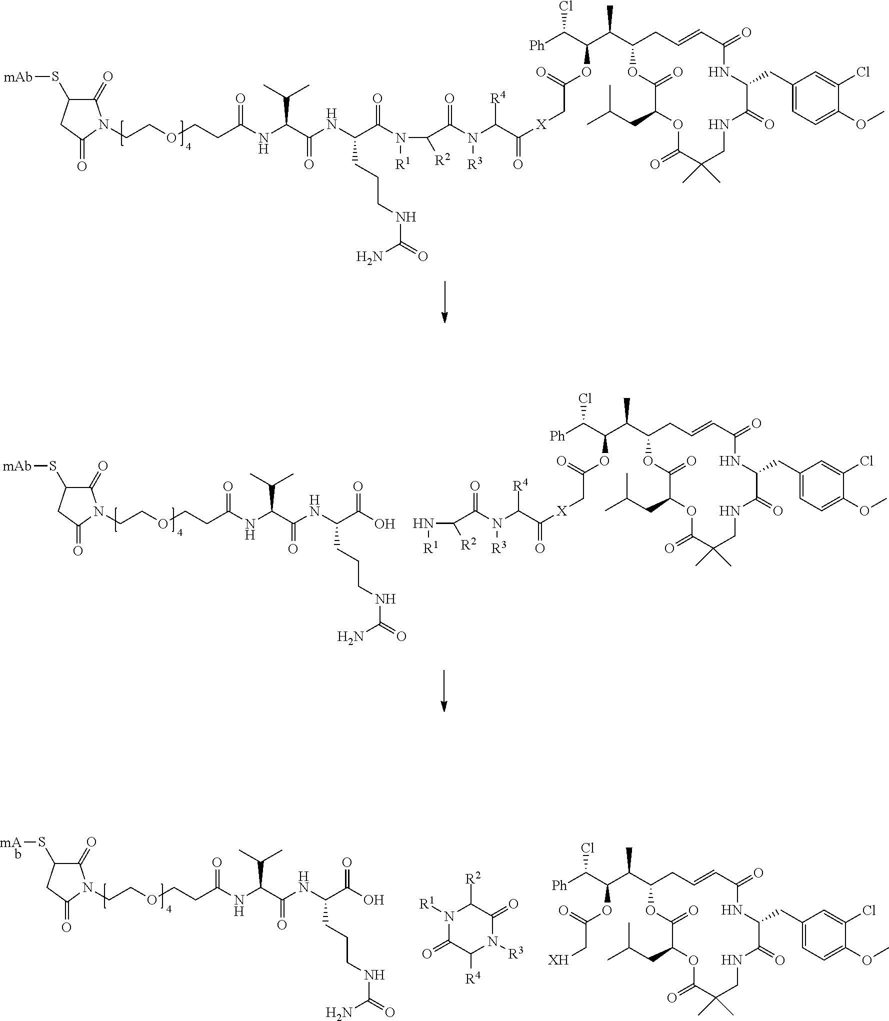

Scheme 1. Cleavage by a protease releases a diketopiperazine and a cryptophycin-55 derivative (e.g. glycinate) (R¹═H, Alkyl, R²=any amino acid side chain, R³═H, (C₁-C₁₀) alkyl, R⁴=any amino acid side chain, X═NH, N(C₁-C₁₀) alkyl, O)

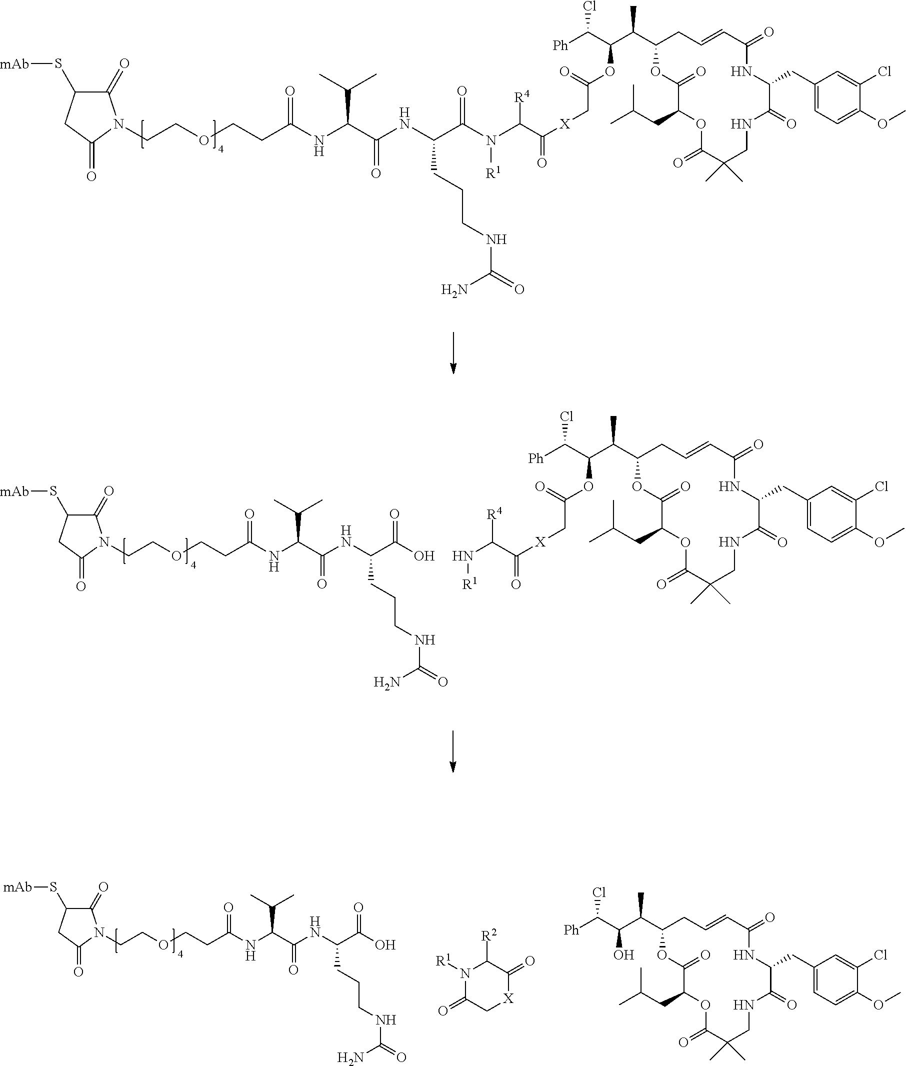

Scheme 2. Cleavage by a protease releases a diketopiperazine (X═NH or N—(C₁-C₁₀) alkyl) or diketomorpholine (X═O) and cryptophycin-55 (R¹═H, (C₁-C₁₀) alkyl, R⁴=any amino acid side chain, X═NH, N(C₁-C₁₀) alkyl, O)

The conjugate of the present invention can be prepared according to conventional techniques well known in the art.

For example, Cryptophycin-52 can be prepared according to a route published by Weiss C, et al (2012) Beilstein Journal of Organic Chemistry 8:2060-6 and Weiss C, et al (2013) Natural Product Reports 30:924-40.

Cryptophycin-55 can be prepared starting from Cryptophycin-52, for example by opening the oxirane ring of Cryptophycin-52. Opening of an oxirane ring in order to introduce a halogen atom is well within the knowledge of the skilled person. For example hydrochloric acid can be used. Reaction media are usually organic solvents suitable for this kind of reactions, as well as reaction conditions and working up of the reaction and isolation of the desired product.

Cryptophycin-55-glycinate can also be prepared according to well-known synthetic methods, for example through the corresponding trifluoroacetate (see Liang J, et al (2005) Investigational New Drugs 213-224).

Self-immolative linkers suitable for the present invention are also well-known as well as their preparation.

In a preferred embodiment of the present invention, tert-Butyl-15-hydroxy-4,7,10,13-tetraoxapentadecanoate can be synthesized according to a procedure published in Seitz O, et al (1997) Journal of Organic Chemistry 62:813-826. tert-Butyl-15-maleimido-4,7,10,13-tetraoxapentadecanoate can be synthesized according to Warnecke A, (2002) Dissertation. 15-Maleimido-4,7,10,13-tetraoxapentadecanoic acid can be prepared according to Warnecke A, (2002) Dissertation.

In a preferred embodiment of the present invention, maleimide-PEG-Val-Cit-Pro-Gly can be synthesized according to standard Fmoc SPPS [Fields G and Noble R L, (2009) International Journal of Peptide and Protein Research 33, 3:161-214].

Other well-known self-immolative linkers can be used in the present invention.

Thereafter, Cryptophycin-55-glycinate Peptide can be prepared by the well-known Maleimide conjugation method.

An antibody-drug conjugate (ADC) with cryptophycin-55-glycinate can be prepared according the common knowledge in this field. For example upon partial reduction of the antibody inter-chain disulfide bonds with TCEP (tris(2-carboxyethyl)phosphine) method or the partial reduction of the antibody inter-chain disulfide bonds with 2-MEA (2-Mercaptoethylamine.HCl) method.

Any other suitable method can be conveniently used.

Peptide-drug conjugates (PDC) according to the present invention can also be prepared with conventional methods.

ADCs and PDCs of the present invention can be tested for their efficacy by in vitro, for example on selected cell lines recognizing the antibody, for ADCs, or presenting receptors recognized by the peptide for PDCs, or in vivo methods using laboratory animal models.

The cryptophycin-55-glycinate ADC according to the present invention shows a much higher cytotoxicity than Trastuzumab on a Her2-positive breast carcinoma cell line (SK-BR₃).

Different from cryptophycin-55 and cryptophycin-55-glycinate, the ADC cytotoxic effect is highly selective for the HER2-positive SK-BR3 cell line compared to MCF7, a breast carcinoma cell line with low HER2-expression or HCT116, a colon carcinoma cell line with low HER2-expression.

The cryptophycin-based conjugates according to the present invention offer several advantages:

The cryptophycin derivatives released show cytotoxic effects towards tumor cells already at nanomolar or subnanomolar concentration.

The cryptophycin derivatives released are not a substrate for PgP and therefore resistance against cryptophycins may be lower.

The synthetic route is based on cryptophycin-55-glycinate, a cryptophycin which does not have to undergo chemical modification on its backbone before conjugation

Selective addressing of HER2-overexpressing breast tumor cells is possible via the antibody

The ADC offers an alternative mechanism of drug-liberation with formation of diketopiperazine derivatives or analogues after enzymatic cleavage of the linker

The cryptophycin derivatives can be used for synthesizing different conjugates, thus providing higher diversity of drug conjugates.

The conjugates according to the present invention can be administered by means of a pharmaceutical composition, wherein an effective amount of the conjugate is admixed with at least one pharmaceutically acceptable vehicle and/or at least one pharmaceutically acceptable excipient.

Said compositions are suitable for veterinary or human administration.

The compositions of the present invention can be in any form that allows for the composition to be administered to a subject, either animal or human. For example, the composition can be in the form of a solid, liquid or gas (aerosol). Typical routes of administration include, without limitation, oral, topical, parenteral, sublingual, rectal, vaginal, ocular, and intranasal. Parenteral administration includes subcutaneous injections, intravenous, intramuscular, intrasternal injection or infusion techniques. Preferably, the compositions are administered parenterally, more preferably intravenously. Pharmaceutical compositions of the invention can be formulated so as to allow a conjugate of the present invention to be bioavailable upon administration. Compositions can take the form of one or more dosage units.

Materials used in preparing the pharmaceutical compositions shall be non-toxic in the amounts used.

The dosage of the active ingredient in the pharmaceutical composition depends on a variety of factors, such as for example, the type of subject to be administered (for example human), the particular form of the conjugate of the Invention, the manner of administration, and the composition employed.

Pharmaceutically acceptable carriers and vehicles are well known in the art and do not need further description. They can be in solid, for example particulate, form or can be liquid, for example, oral syrup or injectable liquid. In addition, the carrier or vehicle can be gaseous, so as to provide an aerosol composition useful in, e. g., inhalatory administration.

When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin, a flavoring agent such as peppermint, methyl salicylate or orange flavoring, and a coloring agent.

When the composition is in the form of a capsule, e. g., a gelatin capsule, it can contain, in addition to materials of the above type, a liquid carrier such as polyethylene glycol, cyclodextrin or a fatty oil.

The composition can be in the form of a liquid, e. g., an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

The liquid compositions of the invention, whether they are solutions, suspensions or other like form, can also include one or more of the following: sterile diluents such as water for injection, saline solution, preferably physiological saline, Ringer's solution, isotonic sodium chloride, fixed oils such as synthetic mono or digylcerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. A parenteral composition can be enclosed in ampoule, a disposable syringe or a multiple-dose vial made of glass, plastic or other material. Physiological saline is a preferred adjuvant.

An injectable composition is preferably sterile.

The amount of the conjugate of the Invention that is effective in the treatment of a particular disorder or condition will depend on the nature of the disorder or condition, and can be determined by standard clinical techniques, In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances.

The composition according to the present invention comprises an effective amount of a conjugate of the invention such that a suitable dosage will be obtained.

The dosage can be determined by body weight or by body surface of the subject to be administered.

Generally, the dosage is typically about 0.1 mg/kg to about 250 mg/kg of the animal's body weight.

In another embodiment, administration can be by direct injection at the site (or former site) of a tumor or the manifestation of an autoimmune disease.

In another embodiment, the conjugates of the invention can be delivered in a vescicle, in particular a liposome (see Langer, Science 249: 1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the conjugates of the invention can be delivered in a controlled release system. In one embodiment, a pump can be used (see Langer, supra; Sefton, CRC Crit. Ref. Biomed. Eng. 14: 201 (1987); Buchwald et al., Surgery 88: 507 (1980); Saudek et al., N. Engl. J. Med. 321: 574 (1989)). In another embodiment, polymeric materials can be used (see Medical Applications of Controlled Release, Langer and Wise (eds.), CRC Press, Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23: 61 (1983); see also Levy et al., Science 228: 190 (1985); During et al., Ann. Neurol. 25: 351 (1989); Howard et al., J. Neurosurg. 71: 105 (1989)). In yet another embodiment, a controlled-release system can be placed in proximity of the target of the Compounds of the Invention or compositions, e. g., the brain, thus requiring only a fraction of the systemic dose (see, e. g., Goodson, in Medical Applications of Controlled Release, supra, vol. 2, pp. 115-138 (1984)). Other controlled-release systems discussed in the review by Langer (Science 249: 1527-1533 (1990)) can be used.

The term carrier refers to a diluent, adjuvant or excipient, with which a conjugate of the invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to an animal, the Compounds of the Invention or compositions and pharmaceutically acceptable carriers are sterile. Water is a preferred carrier when the Compounds of the Invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, and capsules, capsules containing liquids, powders, sustained-release formulations, suppositories, emulsions, aerosols, sprays, suspensions, or any other form suitable for use.

A general reference can be made to “Remington's Pharmaceutical Sciences” by E. W. Martin.

The following examples further illustrate the present invention.

### EXAMPLE 1

### Preparation of Cryptophycin-55-Glycinate ADC Upon Partial Reduction of the Antibody Inter-Chain Disulfide Bonds with TCEP (tris(2-carboxyethyl)phosphine) Method

From a freshly prepared PBS-EDTA-buffer pH 7.2 a TCEP-PBS-EDTA-solution [3 mM TCEP (Sigma-Aldrich) in 100 uL of PBS-EDTA-solution] and a mAb-PBS-EDTA-solution (9 mg/mL Trastuzumab in 0.8 mL PBS-EDTA-solution; 62 uM Trastuzumab) were prepared. The TCEP-PBS-EDTA-solution was added to the mAb-PBS-EDTA-solution and the resulting solution was incubated at 25° C. for 60 min. Final concentrations of the reagents were as follows: 8 mg/mL reduced Trastuzumab (55 uM), 290 uM TCEP.

Then desalting on a HiTrap Desalting Sephadex G25-5 mL (GE Healthcare/Amersham) column pre-equilibrated in PBS-EDTA-buffer pH 7.2 was performed using an Äkta instrument, and fractions of 0.25 mL volume were collected in PBS-EDTA-buffer. All fractions were tested for their protein-content (2 uL of each fraction was added to 200 μL Bradford-solution). The protein-containing fractions were combined and the antibody concentration was re-determined by Bradford assay resulting 3.3 mg/mL (22.8 uM) with a recovery of 5 mg of total antibody (70% recovery). The concentration of free —SH was determined using Elmann's reagent DTNB (5,5′-dithiobis-(2-nitrobenzoic acid), Sigma-Aldrich/Fluka) and a standard curve with L-Cys (40-50-100-200-400-800 uM). Total —SH concentration was 68.24 uM with an average of three free —SH per antibody.

Partial reduction of inter-chain disulfide bonds was repeated as described above and a solution of Maleimide-PEG-Val-Cit-Pro-Gly-Cryptophycin-55-glycinate (1.5 mM in DMSO; 100 uL) was directly added drop-wise to the sample (900 uL) under continuous agitation for 2 h at 4° C., reaching a final concentration of 150 uM in 10% DMSO, such to obtain a 1:1 molar ratio relative to the free —SH groups and a 3:1 molar ratio relative to the antibody (antibody final concentration was 50 uM, equivalent to 150 uM of total free SH). In parallel a S—S reduced antibody control sample has been left unconjugated as control. At the end of the incubation time, each sample was immediately loaded onto a HiTrap Sephadex G25 column and desalting performed as above. Bradford and Elmann assays were performed on pooled fractions as described above and the results are summarized below.

| | | | | Total | | uM | |
|---|---|---|---|---|---|---|---|
| | | mg/ml | uM | mg | | free | |
| Samples | FXN pools | IgG | IgG | IgG | Yield | SH | SH/IgG |
| ADC | 2-7: 1.25 ml | 3 | 20.9 | 3.75 | 52% | 8.6 | 0.5 |
| C | 7-13: 875 uL | 1.65 | 11.4 | 1.44 | 40% | 30.5 | 2.7 |

Under the selected immunoconjugation reaction conditions the average number of SH/IgG dropped from 2.7 to 0.5, thus suggesting that binding to the drug linker occurred with a drug-antibody ratio (DAR) of about 2.

Then preparative gel exclusion chromatography was performed using an Äkta instrument. 500 uL of the cryptophycin-55-glycinate-conjugated and unconjugated mAb samples were loaded onto a Superdex 200 10/300 GL column (GE Healthcare/Amersham) using PBS-EDTA pH 7.2 as the chromatographic buffer and applying a flow-rate of 0.5 ml/min. 250 uL-fractions were collected. Purification of the drug linker-conjugated antibody held to two protein peaks, peak 2 corresponding to the monomeric form and peak 1 corresponding to an oligomeric form of the cryptophycin-55-glycinate-conjugated antibody, as assessed by comparison with suitable protein MW standards (FIG. 2, left panel). On the contrary, the unconjugated mAb sample resulted in a single peak, corresponding to the monomeric form of the antibody (“mAb control” (FIG. 2, right panel). Peak fractions were pooled and protein concentration determined by Bradford assay. Results are summarized below.

| | | mg/ml | uM | Total mg | |
|---|---|---|---|---|---|
| Samples | FXN pools | IgG | IgG | IgG | Yield |
| Peak 1 | 32-44: 3.25 ml | 0.14 | 0.96 | 0.46 | 53% |
| Peak 2 | 47-55: 2.25 ml | 0.02 | 0.14 | 0.05 | 5.8% |
| Reduced mAb | 46-56: 2.75 ml | 0.34 | 2 | 0.93 | 63% |

### EXAMPLE 2

### Preparation of the Cryptophycin-55-Glycinate ADC Upon Partial Reduction of the Antibody Inter-Chain Disulfide Bonds with 2-MEA (2-Mercaptoethylamine.HCl) Method

From a freshly prepared PBS-EDTA-buffer (137 mM NaCl; 2.7 mM KCl; 10 mM Na₂HPO₄; 2 mM KH₂PO₄; 10 mM EDTA; pH=7.2) a 2-MEA-PBS-EDTA-solution (6 mg 2-MEA in 100 uL PBS-EDTA-solution) and a mAb-PBS-EDTA-solution (10 mg Trastuzumab in 1 mL PBS-EDTA-solution; 68.7 uM Trastuzumab) were prepared. The 2-MEA-PBS-EDTA-solution (300 uL) was added to mAb-PBS-EDTA-solution (1 mL) and the resulting solution was incubated at 37° C. for 90 min. Final concentrations of the reagents were as follows: 7.7 mg/mL reduced Trastuzumab (53 uM), 13.8 mg 2-MEA (122 mM).

Then desalting on a HiTrap Desalting Sephadex G25 5 cm column (PBS-EDTA-buffer) was performed and fractions of 0.5 mL volume were collected. All fractions were visually tested for their protein-content (2 uL of each fraction was added to 200 μL Bradford-solution). The protein-containing fractions were combined and a solution of Maleimide-PEG-Val-Cit-Pro-Gly-Cryptophycin-55-glycinate (1.82 mM in DMSO; 150 uL) was added. Incubation at 6° C. for 2 h was followed by concentration of the solution to a volume of less than 1.5 mL. Purification on Äkta Purifier (Superdex 200 highload 16/60 column; PBS-EDTA-buffer) lead to two fractions which were concentrated to mAb-Cry-55-linker-Peak1 (1.54 μg/μL) and mAb-Cry-55-linker-Peak2 (0.32 μg/uL). Peak 1 and 2 corresponded to the two antibody aggregation states described above.

### EXAMPLE 3

Cell Viability Assays

Tumor cell lines (breast carcinoma cell lines SKBR-3 and MCF7 and colon carcinoma cell line HCT116) were obtained from American Type Culture Collection and were grown according to standard protocols. SKBR-3 cells are known to express high Her2 levels and therefore are trastuzumab-sensitive, while MCF7 and HCT116 cells show low Her2 expression and therefore are trastuzumab-resistant. The effects of the cryptophycin-55-glycinate ADC conjugate on tumor cell viability were assessed using the CellTiter-Glo® Luminescent Cell Viability Assay (Promega), according to the manufacturer protocol. To be able to detect a difference between the unconjugated trastuzumab and the trastuzumab ADC, we had previously identify experimental conditions (concentration ranges, incubation time) under which trastuzumab only affected SKBR-3 cell viability by about 30%, while free cryptophycin 55 was equally effective on all tumor cell lines. Cells were plated in black-walled 96-well plates (5000 cells per well for SKBR-3 and MCF7; 2000 cells per well for HCT116) and allowed to adhere overnight at 37° C. in a humidified atmosphere of 5% CO2. Medium was then removed and replaced by fresh culture medium containing increasing concentrations of trastuzumab ADC, unconjugated trastuzumab, or free cryptophycin analogs (CRY-55, CRY55-Gly), and the cells were incubated for 96 h at 37° C. in 5% CO2. Five to eight triplicate datapoints were recorded for each experimental condition. At the end of the incubation luminescence was measured using a Perkin Elmer Wallac 1450 MicroBeta TriLux detector. Compound cytotoxicity was evaluated in comparison to cells treated with PBS (antibodies) or 0.1% DMSO (compounds). CC₅₀ values were calculated by fitting viability data with a four-parameter logistic equation using a Kaleidagraph software. Residual cell viability plots obtained upon treatment of SK-BR3 cells with the different compounds are shown in FIG. 1. The novel cryptophycin-55-glycinate ADC (FIG. 1, lower panels) shows a much higher cytotoxic effect than naked trastuzumab (FIG. 1, upper-right panel) on this Her2-positive breast carcinoma cell line, which is comparable with that shown by cryptophicin-55 and cryptophycin-55-glycinate (FIG. 1, upper-left and -medium panels, respectively). Different from cryptophycin-55 and cryptophycin-55-glycinate, the ADC cytotoxic effect is highly selective for the HER2-positive SK-BR3 cells compared to the Her2-low MCF7 and HCT116 cell lines (Table 1).

TABLE 1
CC₅₀ values

| | Cell line | | |
|---|---|---|---|
| | SK-BR3 | MCF7 | HCT116 |
| Compound | CC₅₀ | CC₅₀ | CC₅₀ |
| Cry-55 | 0.076 nM | 0.18 nM | 0.1 nM |
| Cry-55-Linker | 2.6 nM | 6.1 nM | 4.4 nM |
| Cry-55-Gly | 0.09 nM | 0.25 nM | 0.19 nM |
| Trastuzumab | 0.15 μg/mL | inactive | inactive |
| Trastuzumab reduced | 0.08 μg/mL | inactive | inactive |
| Cry-55-Gly-mAb_Peak1 | 0.01 μg/mL | inactive | inactive |
| Cry-55-Gly-mAb_Peak2 | 0.05 μg/mL | inactive | inactive |

### EXAMPLE 4

### Preparation of Cryptophycin-52

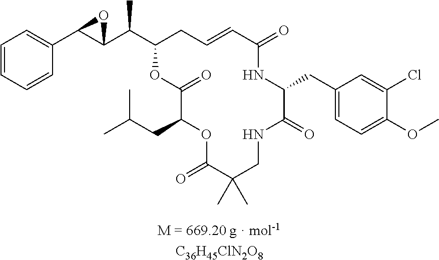 [SMILES: COc1ccc(C[C@H]2NC(=O)/C=C/C[C@@H]([C@H](C)[C@H]3O[C@@H]3c3ccccc3)OC(=O)[C@H](CC(C)C)OC(=O)C(C)(C)CNC2=O)cc1Cl]

Cryptophycin-52 was prepared according to a route published by Weiss C, et al (2012) Beilstein Journal of Organic Chemistry 8:2060-6 and Weiss C, et al (2013) Natural Product Reports 30:924-40.

### Preparation of Cryptophycin-55

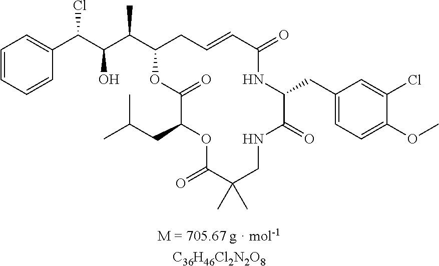 [SMILES: COc1ccc(C[C@H]2NC(=O)/C=C/C[C@@H]([C@H](C)[C@@H](O)[C@@H](Cl)c3ccccc3)OC(=O)[C@H](CC(C)C)OC(=O)C(C)(C)CNC2=O)cc1Cl]

Cryptophycin-52 (40 mg; 60 μmol; 1 eq) was dried in high vacuum and subsequently dissolved in abs. DCM (1 mL). The solution was cooled to −50° C. and HCl (4 M in dioxane; 74.7 μL; 0.30 mmol; 5 eq) was added. After stirring for 2 h the solution was allowed to warm to room temperature (rt) and the volatiles were removed under reduced pressure. Column chromatography (SiO₂; PE/EtOAc 1:3) yielded Cryptophycin-55 (43 mg; 60 μmol; quant.) as colorless solid.

R_{f} (PE/EtOAc 1:3)=0.27;

¹H-NMR (600 MHz, Chloroform-d, TMS) δ [ppm]=7.41-7.34 (m, 5H, uA-C^{ar}H); 7.21 (m, 1H, uC—NH); 7.22 (d, 1H, J=2.2 Hz, uB—C^{2′}H); 7.08 (dd, 1H, J=8.3/2.1 Hz, uB—C^{6′}H); 6.86 (d, 1H, J=8.4 Hz, uB—C^{5′}H); 6.78 (ddd, 1H, J=15.1/10.6/4.4 Hz, uA-C^{β}H); 5.78 (dd, 1H, J=15.1/1.8 Hz, uA-C^{α}H); 5.52 (d, 1H, J=7.9 Hz, uB—NH); 5.16 (ddd, 1H, J=10.6/8.2/2.0 Hz, uA-C^{δ}H); 4.93 (dd, 1H, J=10.2/3.5 Hz, uD-C^{α}H); 4.75 (ddd, 1H, J=7.9/7.7/5.2 Hz, uB—C^{α}H); 4.65 (d, 1H, J=9.6 Hz, uA-C^{η}H); 4.01 (dd, 1H, J=9.7/1.9 Hz, uA-C^{ζ}H); 3.88 (s, 3H, uB—OCH₃); 3.38 (dd, 1H, J=13.4/8.2 Hz, uC—CH^{A}H^{B}NH); 3.18 (dd, 1H, J=13.5/3.8 Hz, uC—CH^{A}H^{B}NH); 3.14 (dd, 1H, J=14.5/5.2 Hz, uB—C^{β}H^{A}H^{B}); 3.06 (dd, 1H, J=14.4/7.6 Hz, uB—C^{β}H^{A}H^{B}); 2.70 (m, 1H, uA-C^{γ}H^{A}H^{B}); 2.49 (m, 1H, uA-C^{ϵ}H); 2.38 (ddd, 1H, J=14.5/10.9/10.9 Hz, uA-C^{γ}H^{A}H^{B}); 1.78 (ddd, 1H, J=14.0/4.5/4.0 Hz, uD-C^{β}H^{A}H^{B}); 1.72 (m, 1H, uD-C^{γ}H); 1.43 (ddd, 1H, J=14.0/8.7/3.5 Hz, uD-C^{β}H^{A}H^{B}); 1.23 (s, 3H, uC—C(CH₃)₂); 1.17 (s, 3H, uC—C(CH₃)₂); 1.04 (d, 3H, J=6.9 Hz, uA-C^{ϵ}HCH₃); 0.93 (d, 3H, J=1.4 Hz, uD-C^{δ}H₃); 0.92 (d, 3H, J=1.2 Hz, uD-C^{δ}H₃).

### Preparation of Cryptophycin-55-Glycinate Trifluoroacetate

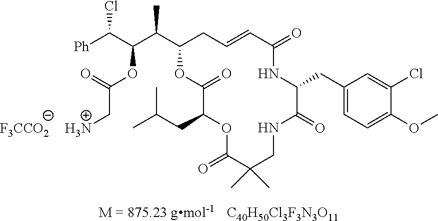 [SMILES: "F3CCO2" ; COc1ccc(C[C@H]2NC(=O)/C=C/C[C@@H]([C@H](C)[C@@H](OC(=O)C[NH3+])[C@@H](Cl)c3ccccc3)OC(=O)[C@H](CC(C)C)OC(=O)C(C)(C)CNC2=O)cc1Cl ; "C40H50Cl3F3N3O11"]

Preparation of Cryptophycin-55-glycinate trifluoroacetate was performed according to Liang J, et al (2005) Investigational New Drugs 213-224 whereas the synthesis was done without purification after the first reaction step. Additionally, the product was not purified with column chromatography but RP-HPLC to yield the Cryptophycin-55-glycinate trifluoroacetate salt.

¹H-NMR (500 MHz, Chloroform-d, TMS) δ [ppm]=7.37-7.32 (m, 3H, uA-C^{ar}H); 7.29 (dd, 2H, J=7.3/2.3 Hz, uA-C^{ar}H); 7.22 (m, 1H, uC—NH); 7.20 (d, 1H, J=2.2 Hz, uB—C^{2′}H); 7.06 (dd, 1H, J=8.4/2.2 Hz, uB—C^{6′}H); 6.84 (d, 1H, J=8.5 Hz, uB—C^{5′}H); 6.60 (bs, 1H, uB—NH); 6.53 (ddd, 1H, J=15.2/10.9/4.2 Hz, uA-C^{β}H); 5.74 (dd, 1H, J=15.3/1.8 Hz, uA-C^{α}H); 5.45 (dm, 1H, J=10.1 Hz, uA-C^{ζ}H); 4.94 (dd, 1H, J=10.4/2.9 Hz, uD-C^{α}H); 4.85-4.79 (m, 2H, uA-C^{δ}H/uA-C^{η}H); 4.54 (ddd, 1H, J=8.1/8.1/5.3 Hz, uB—C^{α}H); 3.87 (s, 3H, uB—OCH₃); 3.63 (d, 1H, J=16.7 Hz, Gly-CH^{A}H^{B}); 3.30 (m, 1H, uC—C^{β}H^{A}); 3.20 (m, 1H, uC—C^{β}H^{B}); 3.16-3.06 (m, 2H, Gly-CH^{A}H^{B}/uB—C^{β}H^{A}); 2.91 (dd, 1H, J=14.5/8.6 Hz, uB—C^{β}H^{B}); 2.64 (m, 1H, uA-C^{ϵ}H); 2.53 (m, 1H, uA-C^{γ}H^{A}); 2.22 (m, 1H, uA-C^{γ}H^{B}); 1.93 (m, 1H, uD-C^{β}H^{A}); 1.70 (m, 1H, uD-C^{γ}H); 1.65 (m, 1H, uD-C^{β}H^{B}); 1.18 (s, 3H, uC—CH₃); 1.12 (s, 3H, uC—CH₃); 1.00 (d, 3H, J=7.2 Hz, uA-C^{ϵ}HCH₃); 0.98 (d, 3H, J=6.7 Hz, CH₃); 0.93 (d, 3H, J=6.5 Hz, CH₃).

Preparation of Self-Immolative Linker

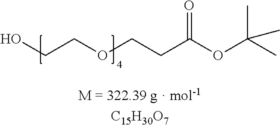

tert-Butyl-15-hydroxy-4,7,10,13-tetraoxapentadecanoate was synthesized according to a procedure published in Seitz O, et al (1997) Journal of Organic Chemistry 62:813-826.

To a solution of tetraethyleneglycol (40.61 mL; 45.64 g; 235 mmol) in abs. THF (125 mL) a piece of sodium (¼ cm) was added. After the sodium had reacted completely, tert-butylacrylate (11.98 mL; 10.57 g; 82.5 mmol) was added dropwise over 20 min and the resulting solution was stirred at rt overnight. The pH was adjusted to 7-8 with NaOH solution (1N) and the solvents were removed in vacuum. The residue was dissolved in sat. NaCl solution (75 mL) and extracted with EtOAc (3×100 mL). The combined organic layers were dried over MgSO₄ and after evaporation of the solvent tert-butyl-15-hydroxy-4,7,10,13-tetraoxapentadecanoate (21.87 g; 67.8 mmol; 82% based on tert-butylacrylate) was obtained as colorless oil.

¹H-NMR (500 MHz, Chloroform-d, TMS) δ [ppm]=3.77-3.57 (m, 18H; OCH₂); 3.01 (bs, 1H, OH); 2.51 (t, 2H, J=6.6 Hz, CH₂COO^{t}Bu); 1.45 (s, 9H, C(CH₃)₃);

¹³C{¹H}-NMR (126 MHz, Chloroform-d, TMS) δ [ppm]=171.1 (COO); 80.7 (C(CH₃)₃); 72.6/70.8/70.7/70.6/70.5/67.0 (OCH₂); 61.9 (HOCH₂); 36.4 (CH₂CO); 28.2 (C(CH₃)₃).

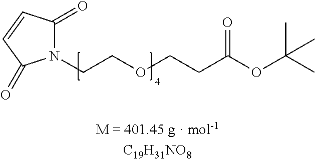

tert-Butyl-15-maleimido-4,7,10,13-tetraoxapentadecanoate was synthesized according to Warnecke A, (2002) Dissertation. Ph₃P (1.09 g; 4.14 mmol; 1 eq) was dissolved in abs. THF (25 mL) and the resulting solution was cooled to −70° C. DIAD (0.8 mL; 4.14 mmol; 1 eq) was added over 1 min and the yellow solution was stirred for 5 min at −70° C. Then tert-butyl-15-hydroxy-4,7,10,13-tetraoxapentadecanoate (2.0 g; 6.20 mmol; 1.5 eq) was added and the solution was stirred for additional 5 min prior to the addition of maleimide (401 mg; 4.14 mmol; 1 eq). After additional stirring for 5 min at −70° C. the cooling bath was removed and the mixture was allowed to warm up to rt. Stirring was continued for 18 h at rt, then the solvent was removed in vacuo and the oily residue was purified by column chromatography (SiO₂; PE/EtOAc 1:3). tert-Butyl-15-maleimido-4,7,10,13-tetraoxapentadecanoate (0.61 g; 1.51 mmol; 36%) was obtained as colorless oil.

R_{f} (PE/EtOAc 1:3)=0.27;

R_{f} (PE/EtOAc 1:1)=0.19;

¹H-NMR (500 MHz, Chloroform-d, TMS) δ [ppm]=6.70 (s, 2H, CH═CH); 3.71 (m, 4H, CH₂); 3.66-3.53 (m, 14H, CH₂); 2.49 (t, 2H, J=6.6 Hz, CH₂COO^{t}Bu); 1.44 (s, 9H, C(CH₃)₃);

¹³C{¹H}-NMR (126 MHz, Chloroform-d, TMS) δ [ppm]=171.0 (CH₂COO^{t}Bu); 170.8 (COCH═CHCO); 134.3 (CH═CH); 80.6 (C(CH₃)₃); 70.8/70.73/70.70/70.65/70.5/70.2/68.0/67.1 (OCH₂); 37.3 (NCH₂); 36.4 (CH₂COO^{t}Bu); 28.3 (C(CH₃)₃).

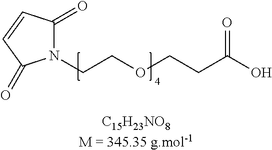

15-Maleimido-4,7,10,13-tetraoxapentadecanoic acid was prepared according to Warnecke A, (2002) Dissertation. tert-Butyl-15-maleimido-4,7,10,13-tetraoxapentadecanoate (1.74 g; 4.33 mmol) was dissolved in abs. DCM (8 mL) and TFA (8 mL) was added. The solution was stirred at rt for 1 h. Next the volatile compounds were removed in vacuo and the residue was again dissolved in DCM (20 mL).

Amberlyst A-21 (6 g) was added to the solution and everything was stirred 1 h at rt. Next the solid was filtered off and the filtrate was concentrated to dryness. 15-Maleimido-4,7,10,13-tetraoxapentadecanoic acid (1.46 g; 4.23 mmol; 98%) was obtained as dark yellow oil.

¹H-NMR (500 MHz, Chloroform-d, TMS) δ [ppm]=9.38 (bs, 1H, COOH); 6.71 (s, 2H, CH═CH); 3.75 (dt, 4H, J=21.6/5.8 Hz, NCH₂/CH₂); 3.68-3.58 (m, 14H, CH₂); 2.63 (t, 2H, J=6.1 Hz, CH₂COOH).

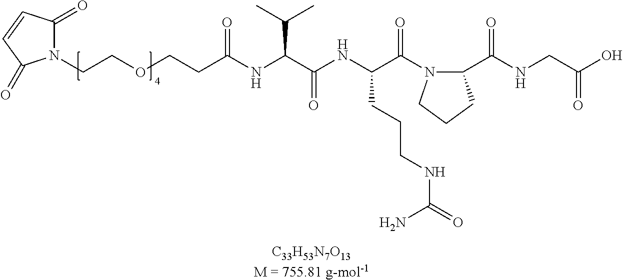

Maleimide-PEG-Val-Cit-Pro-Gly was synthesized according to standard Fmoc SPPS [Fields G and Noble R L, (2009) International Journal of Peptide and Protein Research 33, 3:161-214].

See following Table for details.

| Step | Substance | Volume | Mass | Amount of substance | Equivalents | Reaction time |
|---|---|---|---|---|---|---|
| 1 | Barlos resin | | 1.0 g | | | |
| | Fmoc-Gly | | 1.43 g | 4.8 mmol | 3 eq | |
| | DIPEA | 1.22 mL | 0.93 g | 7.2 mmol | 4.5 eq | |
| | abs. DCM | 5 mL | | | | 3 h |
| 2 | Fmoc-L-Pro | | 1.18 g | 3.48 mmol | 3 eq | |
| | DIPEA | 0.79 mL | 0.60 g | 4.64 mmol | 4 eq | |
| | TBTU | | 1.12 g | 3.48 mmol | 3 eq | |
| | HOBt•H₂O | | 0.47 g | 3.48 mmol | 3 eq | |
| | DMF | 7 mL | | | | 3 h |
| 3 | Fmoc-L-Cit | | 1.38 g | 3.48 mmol | 3 eq | |
| | DIPEA | 0.79 mL | 0.60 g | 4.64 mmol | 4 eq | |
| | TBTU | | 1.12 g | 3.48 mmol | 3 eq | |
| | HOBt•H₂O | | 0.47 g | 3.48 mmol | 3 eq | |
| | DMF | 10 mL | | | | 3 h |
| 4 | Fmoc-L-Cit | | 0.92 g | 2.32 mmol | 2 eq | |
| | DIPEA | 0.53 mL | 0.41 g | 3.13 mmol | 2.7 eq | |
| | TBTU | | 0.75 g | 2.32 mmol | 2 eq | |
| | HOBt•H₂O | | 0.31 g | 2.32 mmol | 2 eq | |
| | DMF | 7 mL | | | | overnight |
| 5 | Fmoc-L-Val | | 1.18 g | 3.48 mmol | 3 eq | |
| | DIPEA | 0.79 mL | 0.60 g | 4.64 mmol | 4 eq | |
| | TBTU | | 1.12 g | 3.48 mmol | 3 eq | |
| | HOBt•H₂O | | 0.47 g | 3.48 mmol | 3 eq | |
| | DMF | 7 mL | | | | 2 h |
| 6 | Maleimide functionalized | | 0.80 g | 2.32 mmol | 2 eq | |
| | PEG-linker | | | | | |
| | DIPEA | 0.79 mL | 0.60 g | 4.64 mmol | 4 eq | |
| | TBTU | | 0.75 g | 2.32 mmol | 2 eq | |
| | HOBt•H₂O | | 0.31 g | 2.32 mmol | 2 eq | |
| | DMF | 7 mL | | | | overnight |

After cleavage from the resin the residue was purified by RP-HPLC and Maleimide-PEG-Val-Cit-Pro-Gly (0.61 g; 0.81 mmol; 70%) was received as colorless lyophilisate.

¹H-NMR (500 MHz, DMSO-d₆) δ [ppm]=12.24 (bs, 1H, COOH); 8.10 (dd, 1H, J=6.1/5.8 Hz, Gly-NH); 8.06 (d, 1H, J=7.3 Hz, Cit-NHCO); 7.81 (d, 1H, J=8.9 Hz, Val-NHCO); 7.02 (s, 2H, CH Maleimide); 6.02 (bm, 1H, Cit-NH); 4.43 (dd, 1H, J=13.4/7.5 Hz, Cit-C^{α}H); 4.31 (dd, 1H, J=8.3/3.9 Hz, Pro-C^{α}H); 4.20 (dd, 1H, J=8.8/6.7 Hz, Val-C^{α}H); 3.78 (dd, 1H, J=17.5/6.1 Hz, Gly-CH^{A}); 3.69 (m, 1H, Pro-C^{γ}H^{A}); 3.68 (dd, 1H, J=17.5/5.8 Hz, Gly-CH^{B}); 3.62-3.53 (m, 5H, PEG-CH₂/Pro-C^{γ}H^{B}); 3.53-3.41 (m, 16H, PEG-CH₂); 3.00-2.90 (m, 2H, Cit-CH₂NH); 2.45 (m, 1H, PEG-CH^{A}H^{B}CONH); 2.34 (m, 1H, PEG-CH^{A}H^{B}CONH); 2.01 (m, 1H, Pro-C^{β}H^{A}); 1.97-1.87 (m, 2H, Val-C^{β}H/Pro-C^{δ}H^{A}); 1.87-1.79 (m, 2H, Pro-C^{β}H^{B}/Pro-C^{γ}H^{B}); 1.67 (m, 1H, Cit-C^{β}H^{A}); 1.49 (m, 1H, Cit-C^{β}H^{B}); 1.44-1.36 (m, 2H, Cit-C^{γ}H₂); 0.81 (d, 3H, J=6.8 Hz, Val-CH₃); 0.79 (d, 3H, J=6.8 Hz, Val-CH₃);

¹³C{¹H}-NMR (126 MHz, DMSO-d₆) δ [ppm]=172.0 (Pro-CO); 171.3 (COOH); 171.0 (Val-CO); 170.9 (Maleimide-CO); 170.1 (Cit-CONH); 170.0 (PEG-CONH); 158.9 (Cit-CONH₂); 134.6 (Maleimide-CH); 69.8/69.77/69.72/69.66/69.5/69.4/67.0 (PEG-CH₂); 59.3 (Pro-C^{α}); 57.1 (Val-C^{α}); 50.2 (Cit-C^{α}); 46.8 (Pro-C^{γ}); 40.6 (Gly-CH₂); 30.7 (Val-C^{β}); 29.2 (Pro-C^{β}); 28.3 (Cit-C^{β}); 26.1 (Cit-C^{γ}); 24.4 (Pro-C^{δ}); 19.2 (Val-C^{γ}); 18.1 (Val-C^{γ});

HRMS (ESI-FT-ICR):

| | |
|---|---|
| Measured [m · z⁻¹] = 778.35962 | [C₃₃H₅₃N₇O₁₃ + Na]⁺ |
| Calculated [m · z⁻¹] = 778.35936 | [C₃₃H₅₃N₇O₁₃ + Na]⁺. |

### Cryptophycin-55-Glycinate Peptide for Maleimide Conjugation

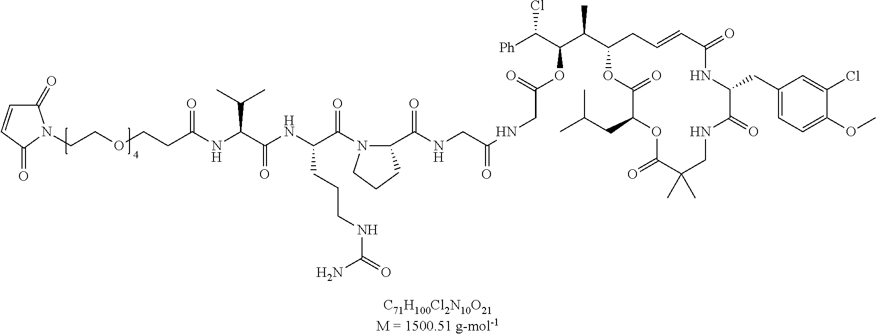

Cryptophycin-55-glycinate trifluoroacetate (10 mg; 13.1 μmol; 1 eq) and Maleimide-PEG-Val-Cit-Pro-Gly (29.7 mg; 39.3 μmol; 3 eq) were dissolved in abs. DMF (0.5 mL). A solution of DIPEA (10 μL; 52.4 μmol; 4 eq), TBTU (12.6 mg; 39.3 μmol; 3 eq) and HOBt.H₂O (5.3 mg; 39.3 μmol; 3 eq) in abs. DMF (0.5 mL) was slowly added. After stirring over night at rt again DIPEA, TBTU and HOBt.H₂O (same amounts as mentioned above) were added. It was stirred again for further 2 h, then the reaction solution directly purified by RP-HPLC. Maleimide-PEG-Val-Cit-Pro-Gly-Cryptophycin-55-glycinate (11.8 mg; 8 μmol; 60%) was achieved as colorless lyophilisate.

HRMS (ESI-FT-ICR):

| | |
|---|---|
| Measured [m · z⁻¹] = 1521.63460 | [C₇₁H₁₀₀Cl₂N₁₀O₂₁ + Na]⁺ |
| Calculated [m · z⁻¹] = 1521.63338 | [C₇₁H₁₀₀Cl₂N₁₀O₂₁ + Na]⁺. |

### EXAMPLE 5

### Synthesis of Cryptophycin 55-Octreotide Conjugate

4-Pentynoyl-octreotide (1):

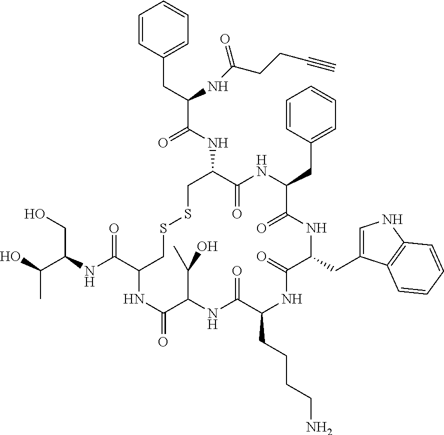 [SMILES: C#CCCC(=O)N[C@H](Cc1ccccc1)C(=O)N[C@H]1CSSCC(C(=O)N[C@H](CO)[C@@H](C)O)NC(=O)C([C@@H](C)O)NC(=O)[C@H](CCCCN)NC(=O)[C@@H](Cc2c[nH]c3ccccc23)NC(=O)[C@H](Cc2ccccc2)NC1=O]

The linear precursor of octreotide was prepared as previously described by Tailhades et al. Angew. Chem. Int. Ed. Engl. 2010, 49, 117. After Fmoc-D-Phe-OH coupling, the Fmoc group was removed with piperidine/DMF (3:7, 2+10 min) followed by washes with DMF (6×1 min) and CH₂Cl₂ (1×1 min). 4-Pentynoic acid (4 eq.) was coupled in presence of Oxyma (4 eq.) and DIC (4 eq.) under stirring at RT for 4 h. Next, the resin was washed with DMF (6×1 min), CH₂Cl₂ (1×1 min) and dried with Et₂O. The resin was cleaved with TFA/PhOH/H₂O/TIS (88:5:5:2) for 2 h at RT. Following TFA evaporation and precipitation with cold Et₂O the peptide was dissolved with H₂O/CH₃CN and lyophilized. The crude peptide was dissolved in a phosphate buffer (pH=7.4, 100 mM, 2 mL per mg of peptide) and the mixture was stirred at RT for 48 h. The peptide was purified by RP-HPLC (Method 1, below) and lyophilized.

HPLC: t_{R}=5.02 min (Method A, below)

MALDI-ToF MS: 1099.5 [M+H]⁺, 1121.6 [M+Na]⁺, 1137.5 [M+K]⁺
PEG Spacer (2 and ₃):

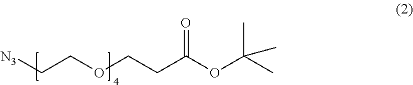

tert-Butyl 15-Azido-4,7,10,13-tetraoxapentadecanoate was synthesized according to Osswald B., (2015) Dissertation (https://pub.uni-bielefeld.de/publication/2733900).

tert-Butyl-15-hydroxy-4,7,10,13-tetraoxapentadecanoate (1.50 g; 4.65 mmol; 1 eq) was dissolved in abs. THF (10 mL) and the resulting solution was cooled to 0° C. Methanesulfonyl chloride (0.54 mL; 6.98 mmol; 1.5 eq) and triethylamine (0.97 mL; 6.98 mmol; 1.5 eq) were added dropwise and the solution was stirred for 30 min at 0° C. then overnight at rt. NaHCO₃ (0.27 g; 3.20 mmol; 0.7 eq) and NaN₃ (0.45 g; 6.98 mmol; 1.5 eq) were added with dist. water (10.5 mL) to the mixture and the resulting solution was stirred for 20 min at rt. THF was removed in vacuum and the remaining solution was stirred at 80° C. for 4 h. After cooling down the mixture was extracted with DCM (3×30 mL), the combined organic layer was dried over MgSO₄ and the solvent was evaporated. The yellowish oily residue was purified by column chromatography (SiO₂; PE/EtOAc 1:1). tert-Butyl 15-Azido-4,7,10,13-tetraoxapentadecanoate (0.74 g; 2.12 mmol; 46%) was obtained as colorless oil.

R_{f} (PE/EtOAc 1:3)=0.69

R_{f} (PE/EtOAc 1:1)=0.4

¹H-NMR (500 MHz, CDCl₃) δ [ppm]=1.44 (s, 9H, C(CH₃)₃), 2.50 (t, 2H, J=6.6 Hz, CH₂COO^{t}Bu), 3.39 (t, 2H, J=5.1 Hz, CH₂N₃), 3.57-3.74 (m, 16H).

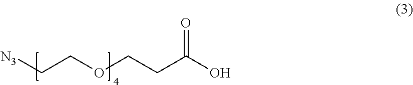

15-Azido-4,7,10,13-tetraoxapentadecanoic acid was prepared according to Warnecke A, (2002) Dissertation.

tert-Butyl 15-Azido-4,7,10,13-tetraoxapentadecanoate (0.22 g; 0.63 mmol) was dissolved in abs. DCM (5 mL) and TFA (5 mL) was added. The solution was stirred at rt for 1.5 h. Next the volatile compounds were removed in vacuo, the residue was dissolved in diethyl ether (10 mL) and the solvent was evaporated (repeated 2×). The solution was concentrated to dryness. 15-Azido 4,7,10,13-Tetraoxapentadecanoic acid (0.18 g; 0.62 mmol; 98%) was obtained as colorless oil.

¹H-NMR (500 MHz, CDCl₃) δ [ppm]=2.63 (t, 2H, J=6.2 Hz, CH₂COOH), 3.39 (t, 2H, J=5.1 Hz, CH₂N₃), 3.58-3.71 (m, 14H), 3.76 (t, 2H, J=6.2 Hz, CH₂CH₂COOH).
Self-immolative linker (4):

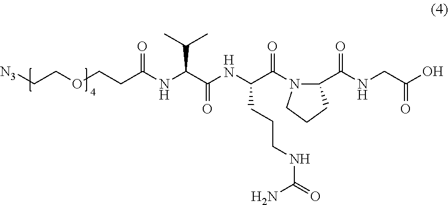

Azide-TEG-Val-Cit-Gly-Pro-OH 4 was synthesized according to standard Fmoc SPPS. See following Table for details.

| Step | Substance | Volume | Mass | Amount of substance | Equivalents | Reaction time |
|---|---|---|---|---|---|---|
| 1 | Barlos resin | | 1.0 g | | | |
| | Fmoc-l-Pro | | 1.62 g | 4.8 mmol | 3 eq | |
| | DIPEA | 1.22 mL | 0.93 g | 7.2 mmol | 4.5 eq | |
| | abs. DCM | 5 mL | | | | 3 h |
| 2 | Fmoc-Gly | | 1.03 g | 3.48 mmol | 3 eq | |
| | DIPEA | 0.79 mL | 0.60 g | 4.64 mmol | 4 eq | |
| | TBTU | | 1.12 g | 3.48 mmol | 3 eq | |
| | HOBt•H₂O | | 0.47 g | 3.48 mmol | 3 eq | |
| | DMF | 7 mL | | | | 3 h |
| 3 | Fmoc-l-Cit | | 1.38 g | 3.48 mmol | 3 eq | |
| | DIPEA | 0.79 mL | 0.60 g | 4.64 mmol | 4 eq | |
| | TBTU | | 1.12 g | 3.48 mmol | 3 eq | |
| | HOBt•H₂O | | 0.47 g | 3.48 mmol | 3 eq | |
| | DMF | 10 mL | | | | 3 h |
| 4 | Fmoc-l-Val | | 1.18 g | 3.48 mmol | 3 eq | |
| | DIPEA | 0.79 mL | 0.60 g | 4.64 mmol | 4 eq | |
| | TBTU | | 1.12 g | 3.48 mmol | 3 eq | |
| | HOBt•H₂O | | 0.47 g | 3.48 mmol | 3 eq | |
| | DMF | 7 mL | | | | 2 h |
| 5 | Azide functionalized | | 0.06 g | 0.21 mmol | 3 eq | |
| | PEG-linker | | | | | |
| | DIPEA | 0.07 mL | 0.05 g | 0.42 mmol | 6 eq | |
| | BOP | | 0.09 g | 0.21 mmol | 3 eq | |
| | DMF | 2 mL | | | | 6 h |

After cleavage from the resin the residue was purified by RP-HPLC and Azide-TEG-Val-Cit-Gly-Pro-OH (0.05 g; 0.07 mmol; 70%) was received as colorless lyophilisate.

MALDI-ToF MS: 702.3 [M+H]⁺, 724.3 [M+Na]⁺
Azide-TEG-Val-Cit-Gly-Pro-Gly-cryptophycin-55 (5):

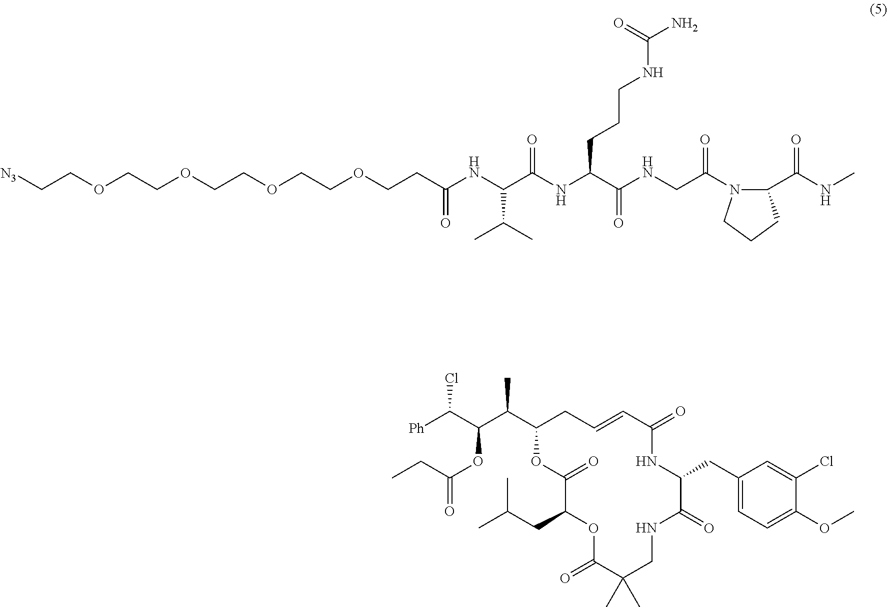

Cryptophycin-55-glycinate trifluoroacetate (5 mg; 5.7 μmol; 1 eq) and Azide-TEG-Val-Cit-Gly-Pro-OH (16.0 mg; 22.8 μmol; 4 eq) were dissolved in abs. DMF (0.5 mL). A solution of DIPEA (5.0 μL; 34.2 μmol; 6 eq), PyBOP (11.9 mg; 22.8 μmol; 4 eq) and HOBt.H₂O (3.5 mg; 22.8 μmol; 4 eq) in abs. DMF (0.5 mL) was slowly added. The reaction mixture was stirred at rt for 3.5 h, then the solution was directly purified by RP-HPLC. Azide-TEG-Val-Cit-Gly-Pro-Cryptophycin-55-glycinate (6.2 mg; 4.3 μmol; 75%) was achieved as colorless lyophilisate.

HPLC: t_{R}=7.06 min (Method A)

MALDI-ToF MS: 1467.6 [M+Na]⁺
Cryptophycin-55-octreotide conjugate (6):

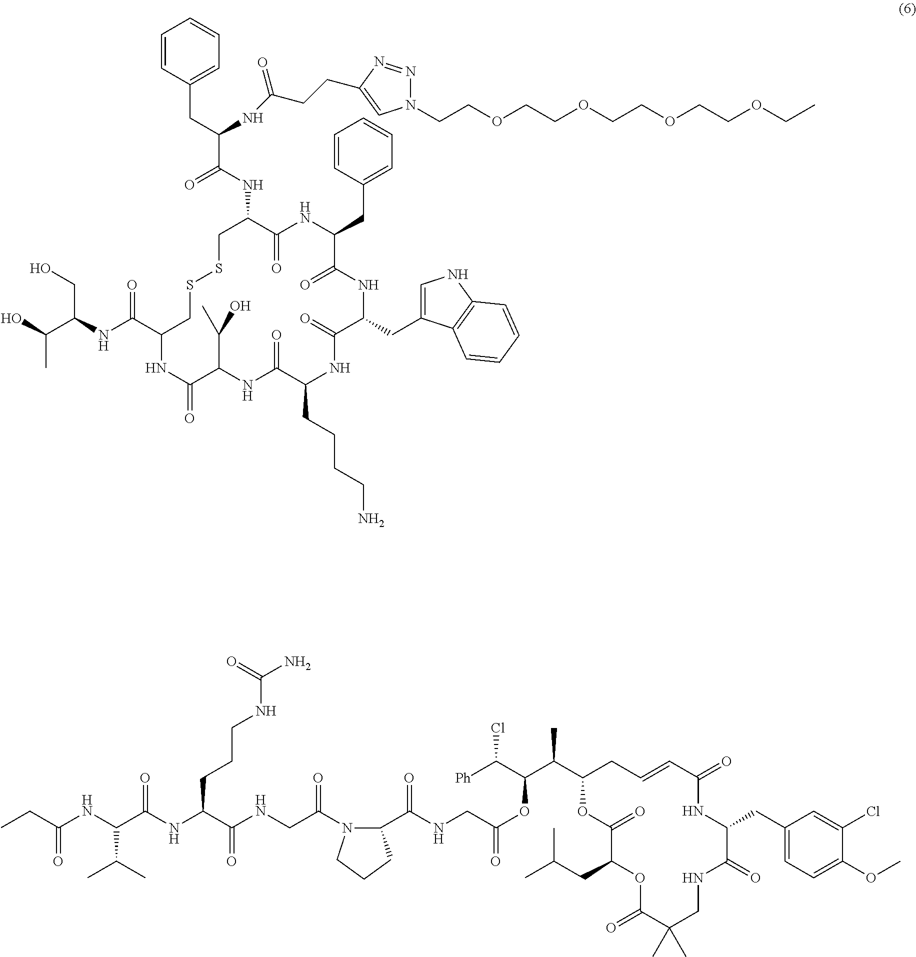

A mixture of the azide-functionalized cryptophycin 5 (1.5 mg, 1.04 μmol, 1.0 eq) and 4-pentynoyl-octreotide 1 (1.14 mg, 1.04 μmol, 1.0 eq) were dissolved in ^{t}BuOH/H₂O (1 mL, 2:1 v/v). Copper powder (2.0 mg) was added and the mixture stirred at RT for 48 h. Afterwards, the reaction mixture was diluted with H₂O/ACN (5 mL, 1:1 v/v) and filtered through Celite®. The filtrate was lyophilized and subsequently purified by RP-HPLC. The cryptophycin-55-octreotide conjugate 6 was obtained as a white solid (0.55 mg, 16% yield).

HPLC: t_{R}=6.79 min (Method A)

ESI-MS (m/z): 2546.6 [M+H]⁺, 1273.7 [M+2H]²⁺, 1284.7 [M+H+Na]²⁺, 1292.7 [M+H+K]²⁺, 862.1 [M+2H+K]⁺

HRMS: Calculated for C₁₂₁H₁₇₀Cl₂N₂₂O₃₀S₂ [M+2H]²⁺ 1272.56304; found 1272.56468
Calculated for C₁₂₁H₁₇₀Cl₂N₂₂O₃₀S₂Na [M+2H+Na]³⁺ 856.03843; found 856.03890

Analytical HPLC:

Analytical HPLC was performed on a THERMO SEPARATION PRODUCTS HPLC unit (controller SN 4000, pump P 4000, autosampler AS 100, detector UV 6000 LP, UV-absorption measured at λ=274 nm), equipped with a PHENOMENEX Jupiter C18 column (dimensions 4.6 mm (ID)×250 mm, grain size 5 μm). The following eluents were used:

Eluent A: H₂O/CH₃CN/TFA 95:5:0.1

Eluent B: H₂O/CH₃CN/TFA 5:95:01

Method A:

Flow rate: 700 μl/min

| | | |
|---|---|---|
| 0 min | 100% A | 0% B |
| 10 min | 0% A | 100% B |
| 11.2 min | 0% A | 100% B |
| 12.7 min | 100% A0% B | |
| 14 min | 100% A0% B | |

Preparative HPLC:

Preparative RP-HPL-chromatography was performed on a MERCK-HITACHI unit (controller: D-7000, pump: L7150, detector: L7420, UV-absorption measured at I=220 nm), equipped with a Macherey-nagel C18-column (dimensions 10 mm (ID)×250 mm, grain size 7 μm).

Method 1 (Eluents A and B as analytical HPLC):

Flow rate: 4 mL/min

| | | |
|---|---|---|
| 0 min | 100% A0% B | |
| 2 min | 100% A | 0% B |
| 35 min | 0% A | 100% B |
| 40 min | 0% A | 100% B |
| 45 min | 100% A0% B | |

ESI-MS:

ESI/APCI mass spectra were recorded using an Esquire 3000 ion trap mass spectrometer (Bruker Daltonik GmbH, Bremen, Germany) equipped with a standard ESI/APCI source. Samples were introduced by direct infusion with a syringe pump. Nitrogen served both as the nebulizer gas and the dry gas. Nitrogen was generated by a Bruker nitrogen generator NGM 11. The spectra shown here are recorded with the Bruker Daltonik esquireNT 5.2 esquireControl software by the accumulation and averaging of several single spectra (as cited). DataAnalysis™ software 3.4 was used for processing the spectra.

HRMS:

Exact ESI mass spectra were recorded in a Thermo-Fisher-Scientific Orbitrap LTQ XL

### EXAMPLE 6

### Synthesis of Cryptophycin 55-Octreotide Conjugate:

Example 5 was repeated but using the following self-immolative linker:

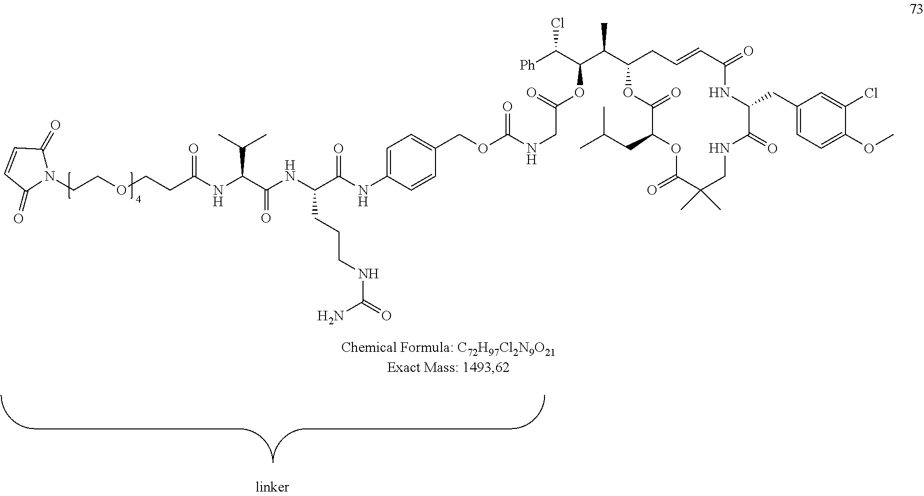

### EXAMPLE 7

RNA Purification and Quantitative RT-PCR

Tumor cell lines (the rat pancreatic tumor cell line AR42J and the human breast carcinoma cell line MCF7) were obtained from the American Type Culture Collection and were grown according to standard protocols.

Total RNA was extracted from cells using the RNeasy Mini Kit (Qiagen, Germantown, Md.) including a DNAse I treatment (Qiagen), following the manufacturers recommendations. One step RT-PCR reactions were assembled in 96-well optical plates. Quantitative RT-PCR was performed in triplicate as follows. Forty uL of a reaction mix were prepared containing 25 uL of 2× Master Mix, 0.5 uL of 100× RT (QuantiTect Probe RT-PCR Kit, Qiagen), and 2.5 uL of each of the following qPCR assays (Applied Biosystems/Thermofisher): rat SSTR2 (Rn01464950_g1), human SSTR2 (Hs00265624_s1), rat GAPDH (Rn01775763_g1), and human GAPDH (Hs04420632_g1). Ten uL of total RNA at concentrations of 3 ng/uL were then added in a final reaction volume of 50 uL, and quantitative RT-PCR was carried out (50° C. 30 min, 95° C. 10 min, followed by 40 cycles: 95° C. 15 sec, 60° C. 1 min) using an ABI 7900 HT real time thermal cycler (Perkin Elmer). Quantitative calculations were performed by using the 2̂(−ΔΔc-t) method on GAPDH-normalized mRNA expression values. The RT-qPCR results, where the SSTR2 expression level in MCF7 cells is expressed as percentage of the corresponding value measured in AR42J cells (set as 100%). The RT-qPCR results confirmed that the AR42J cells express relatively high levels of SSTR2 mRNA, while in MCF7 cells expression levels are much lower (approximately 0.6%).

### EXAMPLE 8

Cell Viability Assays

The effects of the cryptophycin-55-glycinate-octreotide conjugate of Example 5 on the viability of the tumor cell lines AR42J (STTR2^{high}) and MCF7 (SSTR2^{low}) were assessed using the CellTiter-Glo® Luminescent Cell Viability Assay (Promega), according to the manufacturer protocol. Cells were plated in black-walled 96-well plates (5000 cells per well) and allowed to adhere overnight at 37° C. in a humidified atmosphere of 5% CO₂. Medium was then removed and replaced by fresh culture medium containing increasing concentrations of cryptophycin-55-glycinate-octreotide conjugate (CRY55-Gly-Oct), unconjugated octreotide, or free cryptophycin-55-glycinate (CRY55-Gly), and the cells were incubated for 120 h at 37° C. in 5% CO₂. Nine triplicate 1:3 serial dilution datapoints were recorded for each experimental condition (from 0.0015 nM to 10 nM for CRY55-Gly and CRY55-Gly-Oct, from 0.0015 uM to 10 uM for the unconjugated octreotide). At the end of the incubation luminescence was measured using a Perkin Elmer Wallac 1450 MicroBeta TriLux detector. Compound cytotoxicity was evaluated in comparison to cells treated with 0.1% DMSO. IC₅₀ values were calculated by fitting viability data with a four-parameter logistic equation using a Kaleidagraph software. Residual cell viability plots obtained upon treatment of the AR42J and MCF7 cells with the different compounds are shown in FIG. 3 and FIG. 4, respectively. On the STTR2^{high} AR42J tumor cell line the novel cryptophycin-55-glycinate-octreotide conjugate (FIG. 3, upper-left panel) shows a much higher cytotoxic effect than the naked octreotide (FIG. 3, lower panel), with an IC₅₀ value (0.9 nM) very similar to that shown by the cryptophycin-55-glycinate compound (FIG. 3, upper-right panel). On MCF7 the unconjugated octreotide is completely ineffective as expected (FIG. 4, lower panel). Different from what observed on the AR42J cells, the 55-glycinate-octreotide conjugate (FIG. 4, upper-left panel) inhibits MCF7 cell viability with an IC₅₀ value significantly lower (90-fold) than that measured with the cryptophycin-55-glycinate compound (FIG. 4, upper-right panel). In addition, while the cryptophycin-55-glycinate compound inhibits the two cell line viability with similar potencies (compare FIG. 3 and FIG. 4, upper-right panels), the conjugated octreotide cytotoxic effect is quite selective for the STTR2^{high} AR42J cells compared to the SSTR2^{low} MCF7 cells (compare FIG. 4 and FIG. 3, upper-left panels) with IC₅₀ values of 0.9 nM and 27 nM, respectively. IC₅₀ values for all compounds on the two tested cell lines are summarized in the Table

| Cell | Oct-Cry55-Gly | Cry-55-Gly | Octreotide |
|---|---|---|---|
| line | conjugate IC₅₀ | IC₅₀ | IC₅₀ |
| AR42J | 0.9 nM | 0.8 nM | 30% inh. up to 10 uM |
| MCF7 | 27 nM | 0.2 nM | NA |

### EXAMPLE 9

### Activity of Cryptophycin-55-Glycinate and Cryptophycin-55-Glycinate Drug-Linker Molecules on Multidrug Resistant Tumor Cell Lines

H69 cells and the drug-resistant counterpart H69AR were obtained from the American Type Culture Collection and were grown according to standard protocols. The effects of cryptophycin and cryptophycin conjugates on the viability of wild type and drug resistant tumor cell lines were assessed using the CellTiter-Glo® Luminescent Cell Viability Assay (Promega), according to the manufacturer protocol. Cells were plated in black-walled 96-well plates (5000 cells per well) and allowed to adhere overnight at 37° C. in a humidified atmosphere of 5% CO₂. Medium was then removed and replaced by fresh culture medium containing increasing concentrations of cryptophycin analogs or doxorubicin as a control. Cells were incubated with compounds for 120 h at 37° C. in 5% CO₂. Eight triplicate 1:3 serial dilution datapoints were recorded for each experimental condition (from 0.05 nM to 10 nM for CRY55-Gly, CRY55-Gly-LINKER73 and CRY55-Gly-LINKER76, from 1.2 nM to 2700 nM for doxorubicin). At the end of the incubation, luminescence was measured using a Perkin Elmer Wallac 1450 MicroBeta TriLux detector. Compound cytotoxicity was evaluated in comparison to cells treated with 0.1% DMSO.

As shown in the table below and in FIG. 5, cryptophycin or cryptophycin drug linkers showed a similar cytotoxicity on wild type and drug resistant cells, while doxorubicin, used as a control, selectively affected cell viability of wild type cells and had only minor effects on the drug resistant H69AR cell line.

| | H69 cells IC50 | H69AR cells IC50 |
|---|---|---|
| Compound | (nM) | (nM) |
| Cry55 | 0.1 | 0.23 |
| Cry55-gly | 0.09 | 0.15 |
| Example 5 | 1.7 | 3.6 |
| Example 6 | 1.7 | 4.1 |

1. A conjugate of formula (I) 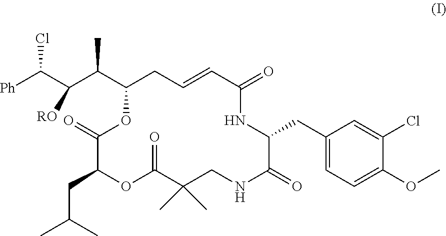
wherein R is —CO—CH₂—X-(A)ₙ-B
wherein X is selected from the group consisting of NR^{a}, wherein R^{a} is selected from a group consisting of H and C₁-C₁₀ alkyl, and O;
A is a self-immolative linker,
n is 0 or 1;
B is selected from the group consisting of a peptide, a polypeptide, a protein, and an antibody,
and pharmaceutically acceptable salts thereof.

2. The conjugate according to claim 1, wherein the antibody is a monoclonal antibody, or a nanobody.

3. The conjugate of claim 2, wherein the nanobody is selected from the group consisting of a single-domain antibody and a camelid antibody.

4. The drug conjugate according to claim 2, wherein B is a monoclonal antibody, and optionally the monoclonal antibody is selected from the group consisting of a trastuzumab, a gemtuzumab, a brentuximab, a rituximab, a cetuximab, a panitumumab, a ofatumumab, a obinutuzumab, and a pertuzumab.

5. The conjugate according to claim 1, wherein B is a peptide, and optionally the peptide binds to a somatostatin receptor.

6. The conjugate according to claim 5, where the peptide is selected from the group consisting of an octreotide, a pasireotide and a lanreotide.

7. The conjugate according to claim 1, wherein the self-immolative linker A is a moiety of formula (II) 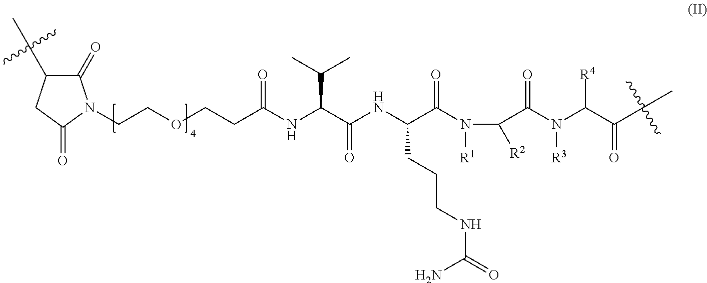
wherein R¹ is selected from the group consisting of H, and (C₁-C₁₀) alkyl,
and R², optionally together with R¹, is the residue of an amino acid side chain,
and R³ is selected from the group consisting of H, and (C₁-C₁₀) alkyl,
and R⁴, optionally together with R³, is the residue of an amino acid side chain.

8. The conjugate according to claim 1, wherein the self-immolative linker A is selected from the group consisting of

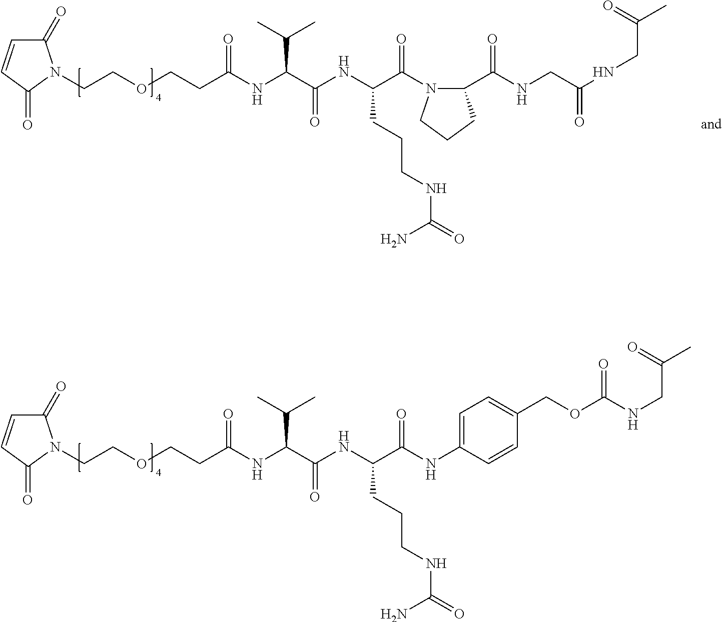

9. The conjugate according to claim 7, having a formula (III) 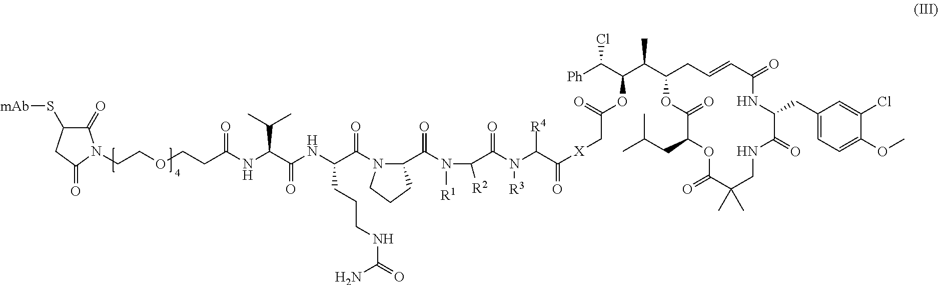
wherein X is selected from the group consisting of NH, N—(C₁-C₁₀)— Alkyl, and O;
and mAb is a monoclonal antibody or a nanobody, or a peptide or a polypeptide.

10. The conjugate according to claim 7 having a formula (IV),

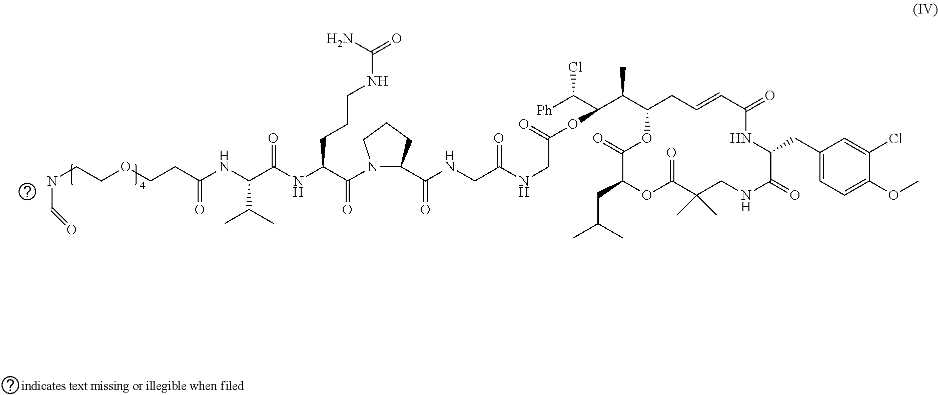

11. The conjugate according to claim 1 having a formula (V) 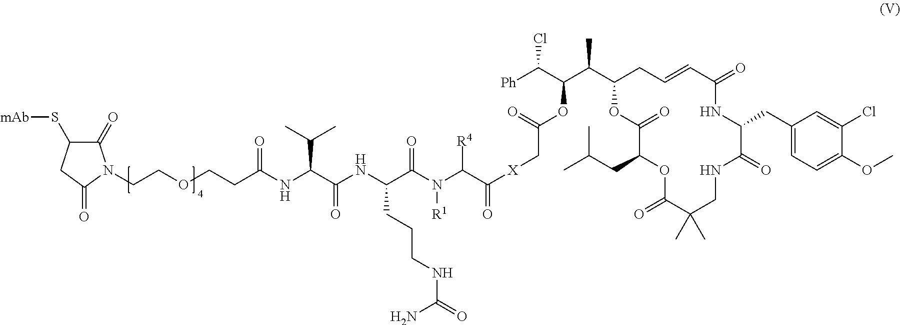
wherein R¹ is selected from the group consisting of H, and (C₁-C₁₀) alkyl,
and R⁴, optionally together with R¹, is the residue of an amino acid side chain;
and X is selected from the group consisting of NH, N—(C₁-C₁₀), Alkyl and O;
and mAb is a monoclonal antibody, a peptide or a polypeptide.

12. The conjugate according to claim 6 having a formula (VI)

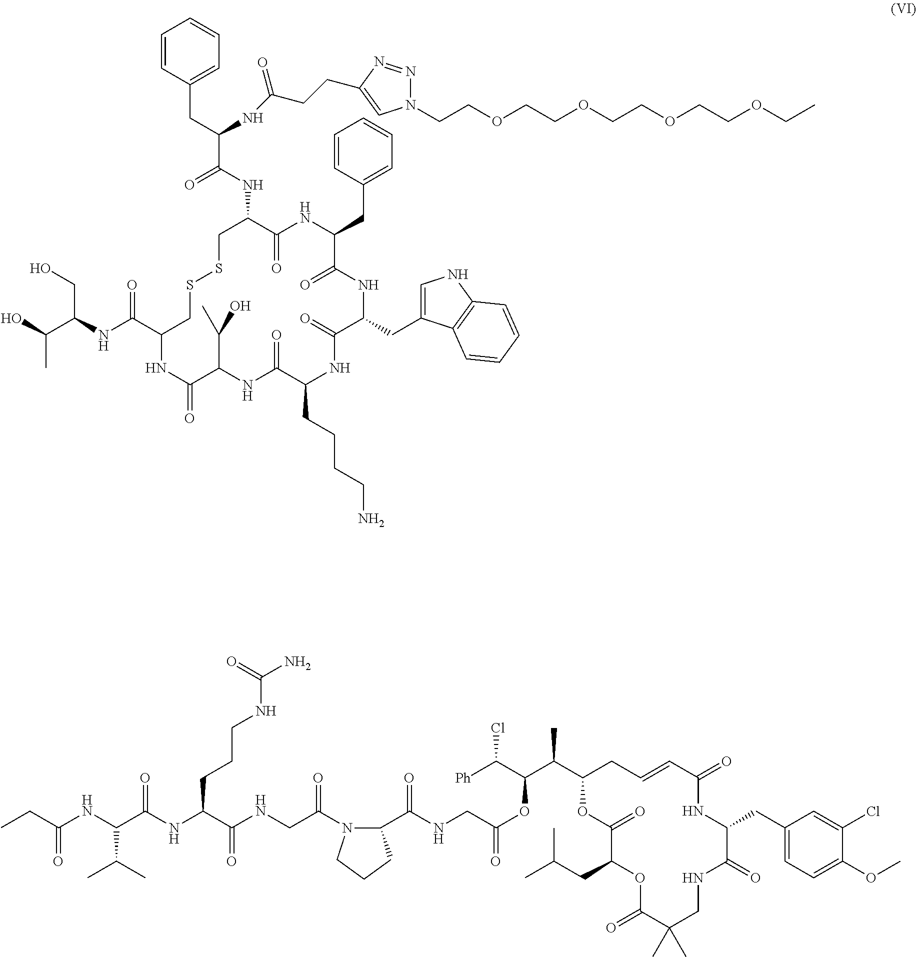

13. A pharmaceutical composition comprising a conjugate of claim 1 as active ingredient in admixture with at least one pharmaceutically acceptable vehicle and/or excipient.

14. The conjugate of claim 1 formulated for use as a medicament.

15. The conjugate of claim 14, formulated as a medicament for the therapeutic treatment of a disease selected from the group consisting of a cancer, an autoimmune disease and an infective disease.

16. The conjugate of claim 1, formulated for use as a diagnostic.

17. The conjugate according to claim 16, formulated for as a diagnostic for the diagnosis of a disease selected from the group consisting of a cancer, an autoimmune disease and an infective disease.

18. A method for treating a cancer, an autoimmune disease or an infective disease, comprising administering to an individual in need thereof an effective amount of a pharmaceutical composition of claim 13.

19. A method for diagnosing a cancer, an autoimmune disease or an infective disease, comprising use of an effective amount of a conjugate of claim 1.
